(19) United States
(12) Patent Application Publication
(10) Pub. No.: US 2014/0142863 A1
(43) Pub. Date: May 22, 2014

(54) Methods And Systems For Orthopedic And Bone Disease Treatment

(71) Applicant: University of Iowa Research Foundation, Iowa City, IA (US)

(72) Inventors: Bruce P. Ayati, Iowa City, IA (US); James A. Martin, Iowa City, IA (US); Sarah A. Holstein, Iowa City, IA (US)

(21) Appl. No.: 14/086,596

(22) Filed: Nov. 21, 2013

Related U.S. Application Data
(60) Provisional application No. 61/729,391, filed on Nov. 22, 2012.

(51) Int. Cl. A61B 5/00

(52) U.S. Cl. CPC A61B 5/4504
USPC 702/19

(57) ABSTRACT

Methods and systems for orthopedic treatment and bone disease treatment are disclosed. An example method can comprise creating a level set function, receiving bone marrow biopsy data, determining a plurality of parameters of the level set function based on the bone marrow biopsy data, determining a bone marrow interface, and predicting bone mass, bone volume, cell counts and spatial distributions for one or more of, osteoclasts, osteoblasts, pre-osteoblasts, osteocytes, plasma cells, stromal cells, and tumor cells. The disclosed methods and systems can predict treatment outcome of a bone disease for a specific patient. In an aspect, the disclosed methods and systems can incorporate cellular interactions implicitly and therefore do not need to refer explicitly to any of the biochemistry in the system.

### CROSS REFERENCE TO RELATED APPLICATIONS

The present application claims the priority benefit under 35 U.S.C. §119(e) of U.S. Provisional Application Ser. No. 61/729,391, filed Nov. 22, 2012, the contents of which are herein incorporated by reference.

### GOVERNMENT SUPPORT CLAUSE

This invention was made with government support under award DMS-0914514 awarded by the National Science Foundation and under grant P-50 AR055533 awarded by the National Institutes of Health. The government has certain rights in the claimed invention.

### BACKGROUND

Bone diseases such as multiple myeloma are incurable diseases that can lead to a debilitating derangement in bone homeostasis. Bone marrow biopsy cores are rich in data concerning the state of a bone disease. However, little is known about how to correlate observed morphological features in bone marrow biopsy data with clinical course of a bone disease such as multiple myeloma with respect to the development of skeletal-related events or the response to a treatment. Furthermore, little attention has been paid to spatial relationships observed amongst plasma cells, osteoclasts, osteoblasts, osteocytes, and stromal cells in bone marrow biopsy data. These and other shortcomings are addressed by the present disclosure.

### SUMMARY

It is to be understood that both the following general description and the following detailed description are exemplary and explanatory only and are not restrictive, as claimed. In an aspect, methods and systems for orthopedic treatment and bone disease treatment are disclosed. Specifically, one or more level set functions can be created for bone remodeling, and bone marrow biopsy data can be analyzed to determine a plurality of parameters of one or more level set functions. By applying a specific level set function to the bone marrow biopsy data, the disclosed methods and systems can be used to predict treatment outcome of a bone disease such as multiple myeloma, or orthopedic condition such as articular cartilage damage, and therefore form a basis for medical prognostics. In an aspect, the disclosed methods and systems can incorporate cellular interactions implicitly in one or more level set functions and therefore do not need to refer explicitly to any biochemistry in bone remodeling.

In an aspect, one or more level set functions can be created for a single bone remodeling site involving local interactions between osteoclasts, osteoblasts, pre-osteoblasts, and osteocytes in an individual basic multicellular unit (BMU). An example method can comprise creating a level set function, receiving bone marrow biopsy data, determining a plurality of parameters of the level set function based on the bone marrow biopsy data, and predicting bone volume, bone mass, and cell counts for one or more of, osteoclasts, osteoblasts, pre-osteoblasts, osteocytes, plasma cells, stromal cells, and tumor cells.

In another aspect, one or more level set functions can be created for multiple remodeling sites (e.g., one or more bone marrow biopsy cores) involving spatial interactions between tumor cells, plasma cells, stromal cells, osteoclasts, osteoblasts, pre-osteoblasts, and osteocytes. An example method can comprise creating a level set function, receiving bone marrow biopsy data, determining a plurality of parameters of the level set function based on the bone marrow biopsy data, determining a bone marrow interface, and predicting cell counts and spatial distributions for one or more of, osteoblasts, osteoclasts, stromal cells, plasma cells, and tumor cells. In an aspect, one or more level set functions associated with a single bone remodeling site can be converted to respective level functions associated with multiple remodeling sites.

In an aspect, the bone marrow biopsy data can come from a patient with a bone disease such as multiple myeloma, an orthopedics condition such as articular cartilage damage, or combination thereof. In an aspect, the bone marrow biopsy data can comprise information such as densities and volume fractions (e.g., density per unit area) of specific types of cells (e.g., tumor cells, plasma cells, stromal cells, osteoclasts, osteoblasts, pre-osteoblasts, and osteocytes), spatial relationships between the specific types of cells, bone densities, and morphology of the bone marrow interface.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is a block diagram of an exemplary computing system;

FIG. 2 is a flowchart illustrating an exemplary method for orthopedic treatment and bone disease treatment;

FIG. 3 illustrates interactions between osteocytes, pre-osteoblasts, osteoblasts, and osteoclasts;

FIG. 4 illustrates bone modeling results of level set function (1) using a specific set of parameters;

FIG. 5 illustrates dynamics of bone volume during a single event of a targeted bone remodeling;

FIG. 6 illustrates steady state bone volume, Zₛₛ, as a simultaneous function of the effectiveness of osteocyte paracrine signaling on stromal cell differentiation g₂₁, and pre-osteoblast autocrine signaling for pre-osteoblast proliferation g₃₂;

FIG. 7 illustrates steady state bone volume, Zₛₛ, computed as a function of effectiveness of osteocyte paracrine signaling on stromal cell differentiation, g₂₁, with all other parameters held at baseline values;

FIG. 8 illustrates that the parameter g₂₁ can be considerably more sensitive to variation than g₃₂;

FIG. 9 illustrates change in bone mass as a function of time in a simulated pathological remodeling;

FIG. 10 illustrates bone remodeling results obtained by including treatment with an anti-sclerostin drug, implemented by modifying the parameters g₂₂ and g₄₄;

FIG. 11 illustrates an example model of local “microenvironment” interactions;

FIG. 12 illustrates bone modeling results of level set function (2) in the no tumor case;

FIG. 13 illustrates bone modeling results of level set function (2) with multiple myeloma dysregulated autocrine signaling;

FIG. 14 is a flowchart illustrating another example method for orthopedic treatment and bone disease treatment;

FIG. 15 illustrates aggregation across space of the dynamics under normal bone remodeling (r₁₁=r₁₂=r₂₁=r₂₂=0) of the spatially explicit model;

FIG. 16 illustrates bone marrow interface snapshots in time under normal bone remodeling;

FIG. 17 illustrates aggregation across space of the dynamics under multiple myeloma dysregulated bone remodeling (r₁₁=0.005, r₁₂=0, r₂₁=0, r₂₂=0.2) of the level set function (3);

FIG. 18 illustrates bone marrow interface snapshots in time under multiple myeloma dysregulated bone remodeling; and

FIG. 19 is a flowchart illustrating another exemplary method for orthopedic treatment and bone disease treatment.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

In an aspect, methods and systems for bone remodeling are disclosed. As an example, a level set function can be created for bone remodeling, and bone marrow biopsy data can be analyzed to determine a plurality of parameters of the level set function. By applying the level set function to bone marrow biopsy data, bone volume, bone mass, cell counts and spatial distributions for one or more of, osteoclasts, osteoblasts, and tumor cells can be predicted. The disclosed methods and systems can be used to predict treatment outcome of a bone disease such as multiple myeloma, or an orthopedic condition such as articular cartilage damage, or combination thereof, therefore form a basis for medical prognostics.

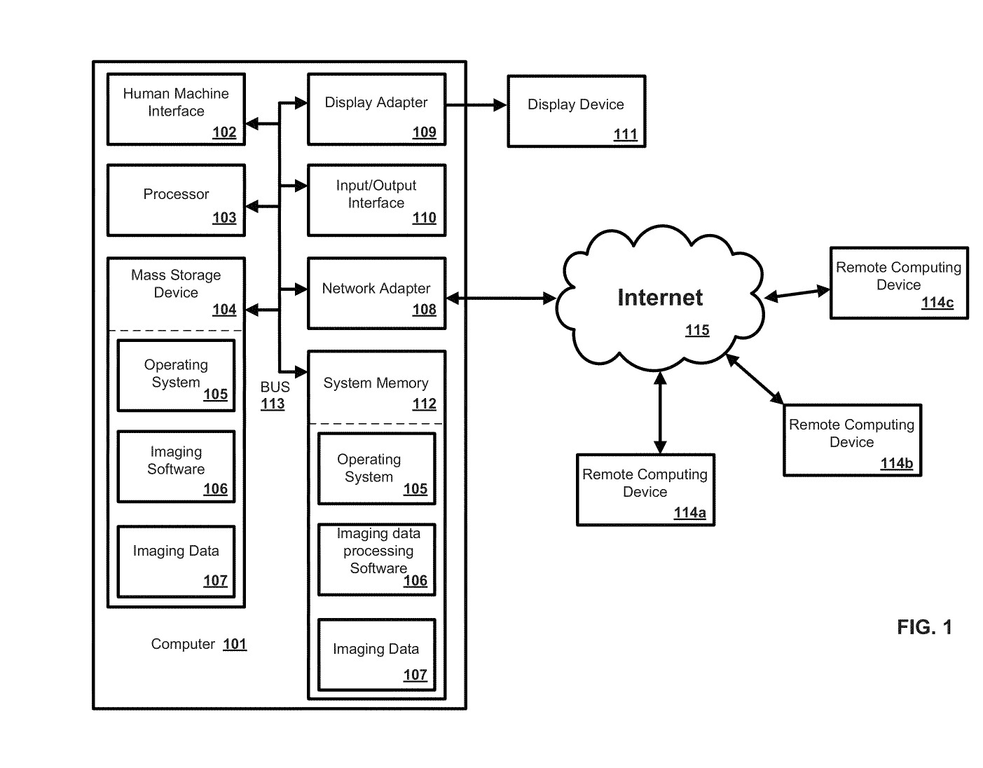

One skilled in the art will appreciate that provided is a functional description and that the respective functions can be performed by software, hardware, or a combination of software and hardware. The methods can comprise the imaging data processing software 106 as illustrated in FIG. 1 and described below. In one exemplary aspect, the units can comprise a computer 101 as illustrated in FIG. 1 and described below.

FIG. 1 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 101. The components of the computer 101 can comprise, but are not limited to, one or more processors or processing units 103, a system memory 112, and a system bus 113 that couples various system components including the processor 103 to the system memory 112. In the case of multiple processing units 103, the system can utilize parallel computing.

The system bus 113 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 113, and all buses specified in this description can also be implemented over a wired or wireless network connection and each of the subsystems, including the processor 103, a mass storage device 104, an operating system 105, imaging data processing software 106, imaging data 107, a network adapter 108, system memory 112, an Input/Output Interface 110, a display adapter 109, a display device 111, and a human machine interface 102, can be contained within one or more remote computing devices 114a,b,c at physically separate locations, connected through buses of this form, in effect implementing a fully distributed system.

The computer 101 typically comprises a variety of computer readable media. Exemplary readable media can be any available media that is accessible by the computer 101 and comprises, for example and not meant to be limiting, both volatile and non-volatile media, removable and non-removable media. The system memory 112 comprises computer readable media in the form of volatile memory, such as random access memory (RAM), and/or non-volatile memory, such as read only memory (ROM). The system memory 112 typically contains data such as imaging data 107 and/or program modules such as operating system 105 and imaging data processing software 106 that are immediately accessible to and/or are presently operated on by the processing unit 103.

In another aspect, the computer 101 can also comprise other removable/non-removable, volatile/non-volatile computer storage media. By way of example, FIG. 1 illustrates a mass storage device 104 which can provide non-volatile storage of computer code, computer readable instructions, data structures, program modules, and other data for the computer 101. For example and not meant to be limiting, a mass storage device 104 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 104, including by way of example, an operating system 105 and imaging data processing software 106. Each of the operating system 105 and imaging data processing software 106 (or some combination thereof) can comprise elements of the programming and the imaging data processing software 106. As an example, imaging data processing software 106 can be used to process imaging data 107, to determine parameters for a level set function created for bone modeling. Imaging data 107 can also be stored on the mass storage device 104. As an example, imaging data 107 can comprise bone marrow biopsy data. Imaging data 107 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 101 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the processing unit 103 via a human machine interface 102 that is coupled to the system bus 113, but can be connected by other interface and bus structures, such as a parallel port, game port, an IEEE 1394 Port (also known as a Firewire port), a serial port, or a universal serial bus (USB).

In yet another aspect, a display device 111 can also be connected to the system bus 113 via an interface, such as a display adapter 109. It is contemplated that the computer 101 can have more than one display adapter 109 and the computer 101 can have more than one display device 111. For example, a display device can be a monitor, an LCD (Liquid Crystal Display), or a projector. In addition to the display device 111, other output peripheral devices can comprise components such as speakers (not shown) and a printer (not shown) which can be connected to the computer 101 via Input/Output Interface 110. Any step and/or result of the methods can be output in any form to an output device. Such output can be any form of visual representation, including, but not limited to, textual, graphical, animation, audio, tactile, and the like.

The computer 101 can operate in a networked environment using logical connections to one or more remote computing devices 114a,b,c. By way of example, a remote computing device can be a personal computer, portable computer, a server, a router, a network computer, a peer device or other common network node, and so on. Logical connections between the computer 101 and a remote computing device 114a,b,c can be made via a local area network (LAN) and a general wide area network (WAN). Such network connections can be through a network adapter 108. A network adapter 108 can be implemented in both wired and wireless environments. Such networking environments are conventional and commonplace in offices, enterprise-wide computer networks, intranets, and the Internet 115.

For purposes of illustration, application programs and other executable program components such as the operating system 105 are illustrated herein as discrete blocks, although it is recognized that such programs and components reside at various times in different storage components of the computing device 101, and are executed by the data processor(s) of the computer. An implementation of imaging data processing software 106 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ artificial intelligence (AI) techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

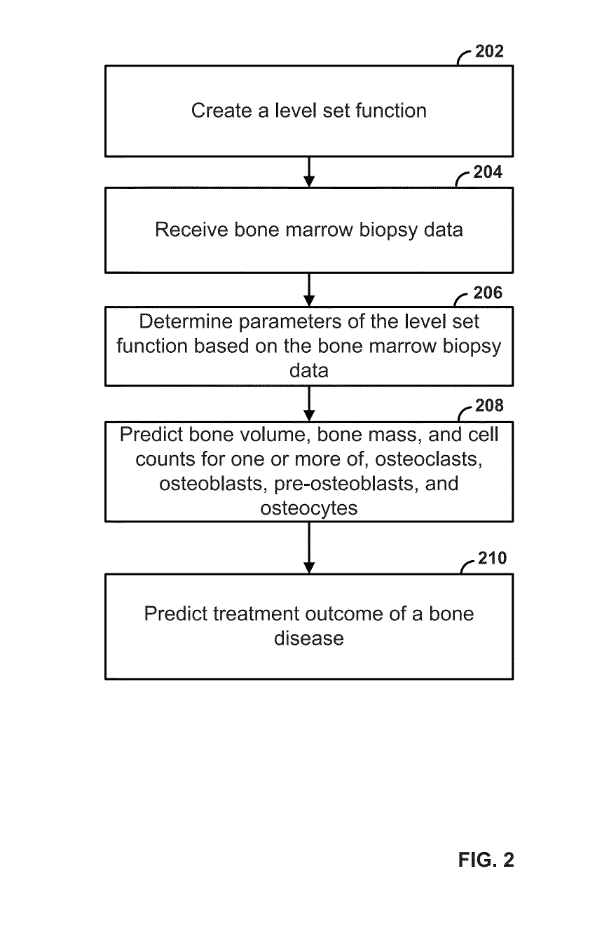

FIG. 2 is a flowchart illustrating an example method for orthopedic treatment and bone disease treatment. At step 202, a level set function can be created. In an aspect, the level set function can be based on mathematical power law formalism of biochemical systems theory (BST). For example, the level set function can be based on mathematical power law formalism of the interactions (e.g., autocrine signaling, paracrine signaling) between osteocytes, pre-osteoblasts, osteoblasts, and osteoclasts. As an example, the level set function can be level set function (1):

$$\begin{matrix} \frac{d S}{d t} = {B}^{{g}_{31}} {{α}_{1} ( 1 - \frac{S}{{K}_{s}} )}_{+} & ( 1 ) \\ \frac{d P}{d t} = {S}^{{g}_{21}} {{α}_{2} ( 1 - \frac{S}{{K}_{s}} )}_{+}^{{g}^{22}} + {P}^{{g}_{32}} {{α}_{3} ( 1 - \frac{S}{{K}_{s}} )}_{+} - {β}_{1} {P}^{{f}_{12}} {C}^{{f}_{14}} - δ P & ( 2 ) \\ \frac{d B}{d t} = {β}_{1} {P}^{{f}_{12}} {C}^{{f}_{14}} - {β}_{2} {B}^{{f}_{23}} - {B}^{{g}_{31}} {{α}_{1} ( 1 - \frac{S}{{K}_{s}} )}_{+} & ( 3 ) \\ \frac{d C}{d t} = {α}_{4} {S}^{{g}_{41}} {{P}^{{g}_{42}} ( ɛ + B )}^{{g}_{43}} {( 1 - \frac{S}{{K}_{s}} )}_{+}^{{g}_{44}} - {β}_{3} {C}^{{f}_{34}} & ( 4 ) \\ \frac{d Z}{d t} = - {k}_{1} C + {k}_{2} B & ( 5 )\end{matrix}$$

wherein C is cell counts for osteoclasts, B is cell counts for osteoblasts, P is cell counts for pre-osteoblasts, S is cell counts for osteocytes, Z is bone volume or bone mass. K_{S} is the number of osteocytes for sclerostin production to inhibit Wnt signaling, α₁ is rate of mature osteoblasts embedded into the extra cellular matrix, α₂ is rate of stem cells differentiate into the pre-osteoblast cell in response to signaling molecules produced by osteocytes, α₃ is pre-osteoblast proliferate rate under the influence of autocrine signaling not inhibited by sclerostin, β₁ is rate of pre-osteoblast cells differentiate into osteoblast cells, δ is apoptosis rate of pre-osteoblast cells, β₂ is apoptosis rate of osteoblast cells, α₄ is differentiation rate of osteoclast precursors, β₃ is rate of pre-osteoblast cells differentiate into osteoblast cells, g₃₁ is effectiveness of osteoblast autocrine signaling, g₂₁ is effectiveness of osteocyte paracrine signaling of pre-osteoblast cells, g₂₂ is effectiveness of sclerostin regulation of osteoblastogenesis, g₃₂ is effectiveness of pre-osteoblast autocrine signaling, f₁₂ is effectiveness of sclerostin regulation of osteoblastogenesis, f₁₄ is effectiveness of pre-osteoblast paracrine signaling of osteoblasts, f₂₃ is effectiveness of osteoblast autocrine signaling for apoptosis, g₄₁ is effectiveness of osteoblast autocrine signaling of osteoclasts, g₄₂ is effectiveness of pre-osteoblast paracrine signaling of osteoclasts, g₄₃ is effectiveness of osteoblast paracrine signaling of osteoclasts, g₄₄ is effectiveness of sclerostin regulation of osteoclastogenesis, f₃₄ is effectiveness of osteoclast autocrine signaling for apoptosis, k₁ is bone resorption rate, and k₂ is bone formation rate. In an aspect, effectiveness can represent strength or efficiency of the pathway. As an example, an effectiveness value of 1 can represent a positive linear response, an effective value of −1 can represent a negative linear response, an effectiveness value of 0.5 or −0.5 can represent a weaker response than linear positive or negative response, an effective value of 2 or −2 can represent a stronger than linear positive or negative response.

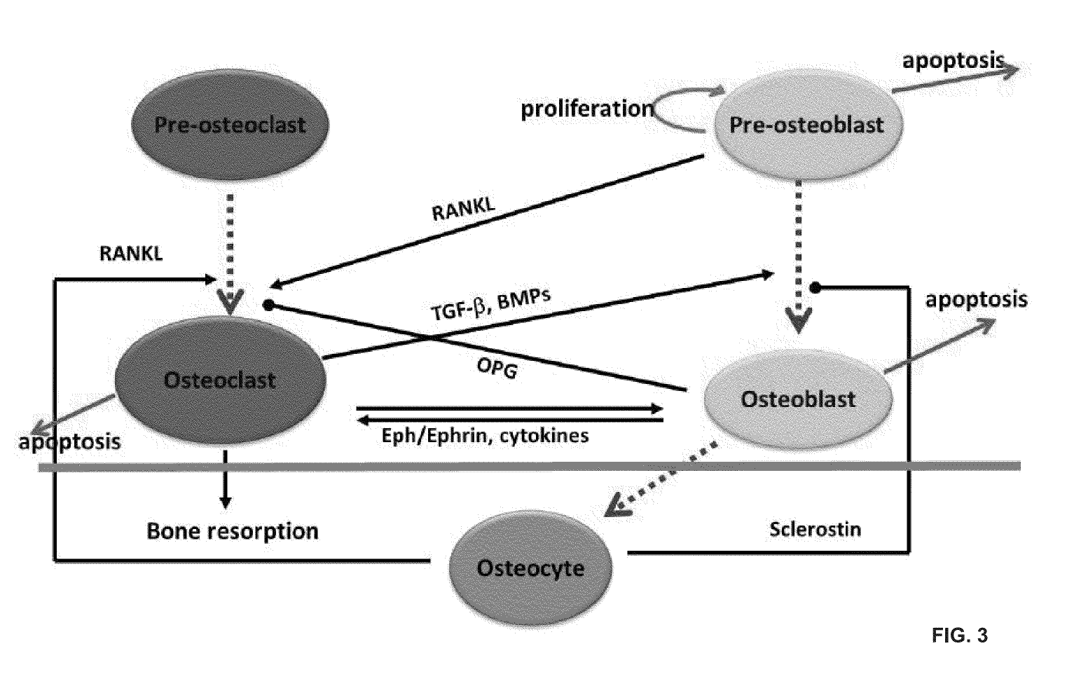

In an aspect, the level set function (1) for bone remodeling can associate cell counts with changes in bone mass, or bone volume, thus allowing a way to track the physical effects of resorption and formation. In another aspect, the level set function (1) for bone remodeling can take into account of interactions between pre-osteoblasts, osteoblasts, and osteoclasts, osteocytes, and their biochemical processes, and thus represent an accurate bone remodeling. FIG. 3 illustrates interactions between osteocytes, pre-osteoblasts, osteoblasts, and osteoclasts. As an example, there can be a large pool of mesenchymal stem cells available to differentiate into pre-osteoblasts. Similarly, it can be assumed there is a large pool of osteoclast progenitor cells available to differentiate to fully committed mature osteoclasts. Such cell differentiation can be determined by autocrine and paracrine signaling. It can be assumed that some percentage of pre-osteoblasts differentiate under the influence of autocrine and paracrine signaling while some percentage undergo apoptosis. It can also be assumed that some percentage of mature osteoblasts will undergo apoptosis, and some percentage of osteoblasts will become embedded in the bone matrix as osteocytes. In an aspect, osteocytes can a play role in the cycle of a targeted bone remodeling, for example, in serving as a source of RANKL to support osteoclastogenesis, and in secreting the bone formation inhibitor sclerostin. Moreover, sclerostin, an osteocyte-secreted bone formation inhibitor, can play a role in regulating local response to changes in a bone microenvironment. As such, the influence of the receptor activator of nuclear factor (RANK)/receptor activator of nuclear factor ligand (RANKL)/osteoprotegerin (OPG) signaling pathway, growth factors such as transforming growth factor (TGF)-β, and other cytokines on bone remodeling cells can be incorporated in the level set function (1).

In an aspect, S(t), or simply S, can denote osteocyte cell counts at given time t. Sclerostin can be produced by osteocytes and inhibit the Wnt/β-catenin pathway. Wnt is known to promote osteoblastic proliferation and differentiation. The effects of sclerostin and the Wnt/β-catenin pathway can be incorporated into the level set function (1) through term

$${( 1 - \frac{S}{{K}_{s}} )}_{+} ,$$

where, (x)₊=max(x, 0) and Kₛ can be a parameter that describes the relation between osteocyte apoptosis and decrease in sclerostin inhibition. In other words, for a threshold level Kₛ of osteocytes, there can be sufficient sclerostin production to inhibit local Wnt signaling. When osteocytes die, the sclerostin level can decrease. This can release osteoblast precursor cells from Wnt inhibition, thereby initiating a cycle of targeted bone remodeling. For example, the Equation (1) of level set function (1) can state that osteocytes can be mature osteoblasts that become embedded in extra cellular matrix at a rate α₁.

In an aspect, pre-osteoblast cell counts at a time t is denoted by P(t), or simply P. Pre-osteoblasts are differentiated mesenchymal stem cells. It can be assumed that the differentiation is controlled by osteocytes through the sclerostin, Wnt/β-catenin pathways, and various growth factors. The effectiveness of sclerostin regulations of the differentiation of mesenchymal stem cells to become pre-osteolbasts can be represented by term

$${( 1 - \frac{S}{{K}_{s}} )}_{+}^{{g}_{22}} ,$$

wherein g₂₂ is a dimensionless parameter. Thus when osteocytes undergo apoptosis due to microdamage, local mesenchymal stem cells can differentiate to pre-osteoblasts. Moreover, the pre-osteoblasts can be free to proliferate and differentiate to mature osteoblasts since they have been released from Wnt inhibition. Equation (2) in the level set function (1) illustrates the dynamics of the pre-osteoblast cell counts. Equation (2) can state that pre-osteoblasts can be differentiated from a large pool of stem cells at a rate α₂ in response to signaling molecules produced by osteocytes, and pre-osteoblasts can proliferate at a rate α₃ under the influence of autocrine signaling provided this is not inhibited by sclerostin. Furthermore, pre-osteoblasts can differentiate to become mature osteoblasts at a rate β₁ under the influence of autocrine and osteoclast regulated paracrine signaling. In an aspect, pre-osteoblasts can undergo apoptosis at a rate δ.

In an aspect, osteoblast cell counts at a time t is denoted by C(t), or simply C. Equation (3) in level set function (1) describes the dynamics of the mature osteoblast cell counts. This equation can state that osteoblasts can be differentiated pre-osteoblasts, that osteoblasts undergo apoptosis, and that some osteoblasts can be embedded in the extra cellular matrix during formation to become osteocytes. In an aspect, the term

$${B}^{{g}_{31}} {{α}_{1} ( 1 - \frac{S}{{K}_{s}} )}_{+}$$

in Equation (3) also appears in equation (1), representing the embedding of osteoblasts that become osteocytes. The term β₁P^{f}^{₁₂}C^{f}^{₁₄} in Equation (3) also appears in equation (2), which can correspond to the differentiation of pre-osteoblasts to become mature osteoblasts. In an aspect, the parameter f₁₂ can describe pre-osteoblast autocrine signaling. The parameter f₁₄ can represent the effects of osteoclast derived paracrine signaling on pre-osteoblasts, which can, in turn, represent the effects of TGF-β on pre-osteoblasts, for example.

In an aspect, osteoclast cell counts at a time t is denoted by C(t), or simply C. The equation (4) in the level set function (1) describes the dynamics of the osteoclast cell counts. Equation (4) can state that mature osteoclasts can come from the differentiation of a large pool pre-osteoclasts at a rate α₄. The differentiation can be influenced essentially by the RANK/RANKL/OPG pathway. Thus the term

$${S}^{{g}_{41}} {{P}^{{g}_{42}} ( ɛ + B )}^{{g}_{43}} {( 1 - \frac{S}{{K}_{s}} )}_{+}^{{g}_{44}}$$

can describe the effects of the RANK/RANKL/OPG pathway. The dimensionless parameter g₄₄ can represent effectiveness of sclerostin regulation of osteoclastogensis. In an aspect, osteocytes can be included as a source of RANKL. The parameter g₄₁ can represent effect of osteocyte derived RANKL signaling on osteoclastogenesis. Pre-osteoblast derived RANKL signaling can be retained via the parameter g₄₂. In an aspect, g₄₁ and g₄₁ can be set to 0. The term (ε+B)^{g}^{₄₃} can represent the effect of OPG acting as a decoy receptor for RANKL. The parameter g₄₃ can take on negative values, and since B has 0 as a steady state value, a sufficiently small number ε relative to the typical cell population can be added to avoid dividing by zero. For example, ε can be set to 1. ε can represent the factor of production when B=0. In an aspect, osteoclast cell death rate can be represented by β₃. As an example, β₃ can be set to 0.1.

In an aspect, Z(t) or simply Z, can denote bone mass or bone volume at a given time t. In an aspect, equation (5) can be developed for a change of bone mass or bone volume depending on the scaling over time. The rate of change of bone mass or bone volume can be:

$$\frac{d Z}{d t} = formation - resorption$$

It can be assumed that the amount of bone being resorbed is proportional to the osteoclast counts, while the amount of bone being formed is proportional to the osteoblast counts. It can also be assumed that the osteocytes can influence the initiation of a targeted basic multicellular unit (BMU) bone remodeling. In an aspect, the set function (1) can be summarized with the following verbal description:
change in osteocytes=increase due to embedded osteoblasts
change in pre-osteoblasts=increase due to differentiation of stromal cells (released from sclerostin or exposure to growth factors)+proliferation of pre-osteoblasts (autocrine signaling of Wnt and growth factors)−differentiation to osteoblasts (growth factors)−apoptosis
change in osteoblasts=increase due to differentiation of pre-osteoblasts (growth factors)−apoptosis−embedding as osteocytes
change in osteoclasts=increase due to differentiation of pre-osteoclasts (due to RANKL and limited by OPG)−apoptosis
change in bone mass=increase due to activity of osteoblasts−activity of osteoclasts.

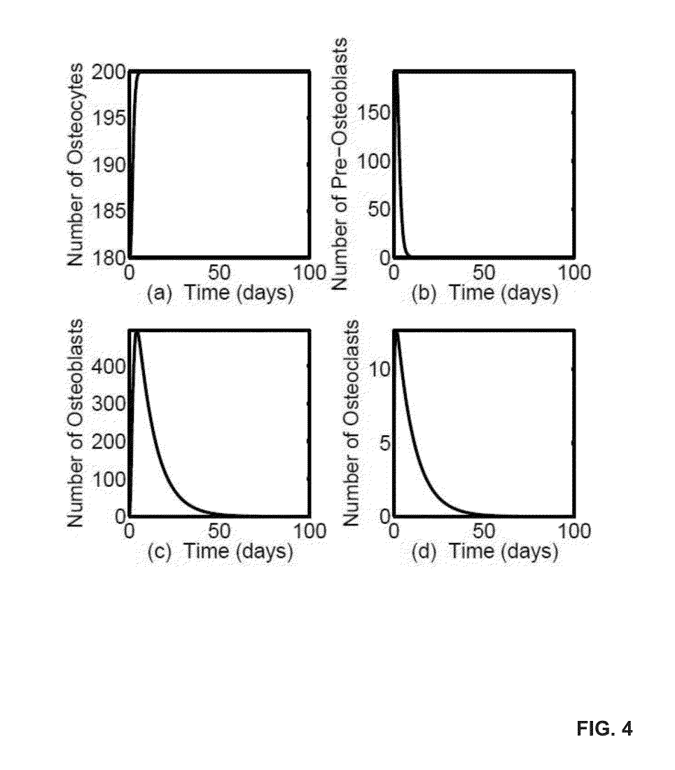

FIG. 4 illustrates results of level set function (1) using a specific set of parameters. In an aspect, MATLAB (e.g., MATLAB ODE suite) can be used for scientific computing of level set function (1). FIG. 4 shows the simulation results for bone cell counts during a single cycle of a normal targeted bone remodeling. In an aspect, a normal targeted bone remodeling can be defined to be a complete remodeling cycle that takes place over a period of one hundred days, in which the amount of new bone formed is equal to the amount of old or damaged bone resorbed. One characteristic of the normal bone remodeling that manifests itself quantitatively is that the steady state value of bone volume, denoted by Zₛₛ, is equal to one hundred percent. As an example, the simulations shown in FIG. 4 result from solving the level set function (1) with parameters K_{S}=200 cells, α₁=0.5 per day, α₂=0.1 per day, α₃=0.1 per day, β₁=0.1 per day, δ=0.1 per day, β₂=0.1 per day, α₄=0.1 per day, β₃=0.1, g₃₁=1, g₂₁=2, g₂₂=1, g₃₂=1, f₁₂=1, f₁₄=1, f₂₃=1, g₄₁=1, g₄₂=1, g₄₃=−1, g₄₄=1, f₃₄=1, ε=1 cell, K₁=0.7% volume per day, K₂=0.015545% volume per day. In an aspect, effectiveness can represent strength or efficiency of the pathway. As an example, an effectiveness value of 1 can represent a positive linear response to the respective component, an effective value of −1 can represent a negative linear response to the respective component, an effective value of 2 can represent a stronger than linear positive response. For example, g₂₁ can indicate a superlinear positive effect of osteocytes on pre-osteoblast generation. It should be noted other numbers for the parameter can be used to fit a remodeling cycle. For example, in a fully functioning prognostic system, the parameters in the level set function can be determined by data from bone marrow biopsy slides, and the system can be updated continuously for different patient types.

FIG. 4(a) shows dynamics of the osteocyte cell counts during an event of targeted bone remodeling. Initially there can be a decrease from the steady state value K_{S} in the osteocyte cell counts, which can correspond to local osteocyte apoptosis, and result in a decrease in local sclerostin levels. This releases stromal cells from sclerostin inhibition, allows for Wnt signaling, and results in proliferation and differentiation of pre-osteoblast cells, as shown in FIG. 4(b). There follows an increase in osteoblast cell number due to differentiation of pre-osteoblasts, and differentiation of osteoclast pre-cursors to mature osteoclasts, as shown in FIG. 4(c) and FIG. 4(d). Bone remodeling ceases once local osteocyte cell counts is replenished, and osteocyte cell network is reestablished, returning sclerostin expression back to sufficient levels thereby inhibiting bone turnover.

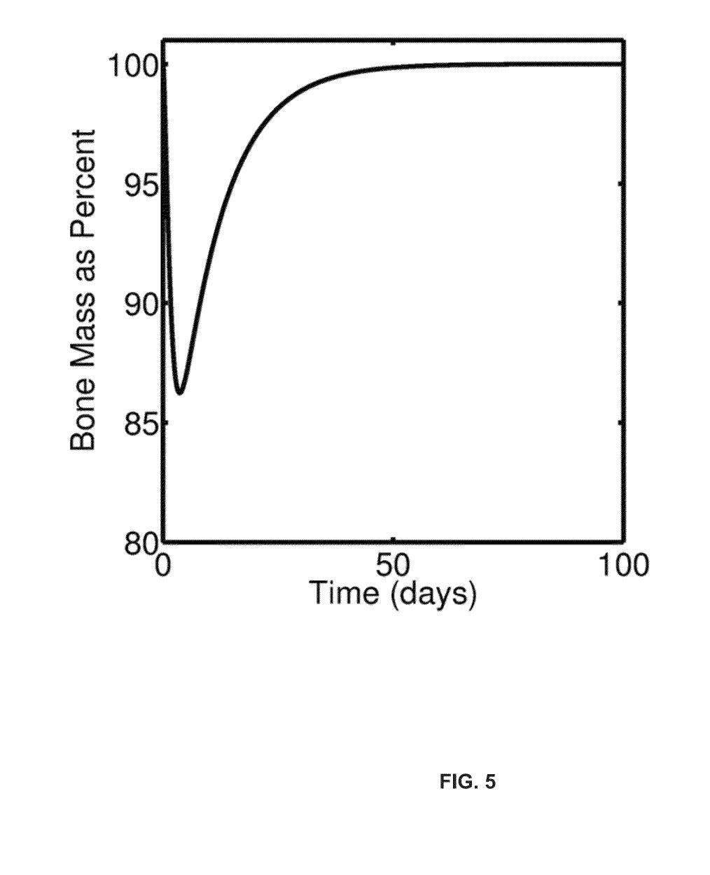

FIG. 5 illustrates dynamics of bone volume during a single event of a targeted bone remodeling. An increase in osteoclast cell numbers can result in bone resorption and the decrease in bone volume, while increase in osteoblast cell number can result in bone formation. In this case, bone turnover can be completely balanced in the sense that the amount of new bone formed equals the amount resorbed. Mathematically this can correspond to a steady state value of bone mass, Zₛₛ˜100%.

In an aspect, one or more parameters of the level set function (e.g., level set function (1) can be associated with role of osteocyte RANKL production. For example, g₄₁ and g₄₂ can correspond to effectiveness of the expression of RANKL by osteocytes, and pre-osteoblasts respectively. Varying value of g₄₁ and g₄₂ simultaneously (e.g., between zero and two) can be associated with how RANKL expression by osteocytes and pre-osteoblasts impacts the regulation of a cycle of targeted bone remodeling. Thus, given cell counts data, the level set function (1) can be used, via parameter fitting, to determine whether osteocytes or pre-osteoblasts can provide dominant RANKL source during a targeted bone remodeling.

In an aspect, one or more parameters of the level set function (e.g., level set function (1)) can be associated with the influence of the effectiveness of osteocyte paracrine signaling and pre-osteoblast autocrine signaling on differentiation of stromal cells into pre-osteoblasts, and proliferation of pre-osteoblast cells. In an aspect, the steady state bone volume, Zₛₛ, as a simultaneous function of the effectiveness of osteocyte paracrine signaling on stromal cell differentiation, g₂₁, and pre-osteoblast autocrine signaling for pre-osteoblast proliferation, g₃₂ can be computed. In an aspect, g₂₁, g₃₂ and Zₛₛ can be considered simultaneously because these terms can influence the production of pre-osteoblasts in Equation (2) of the level set function (1).

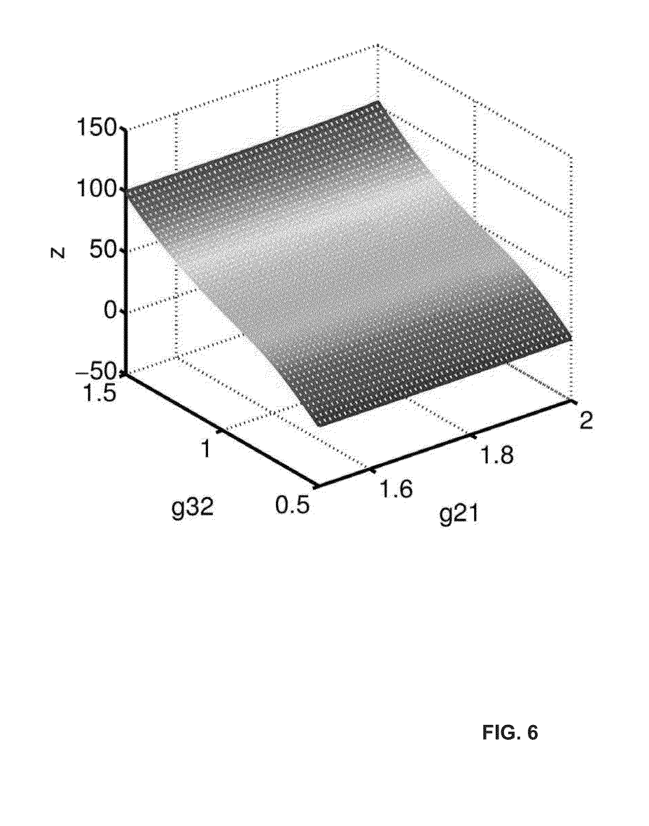

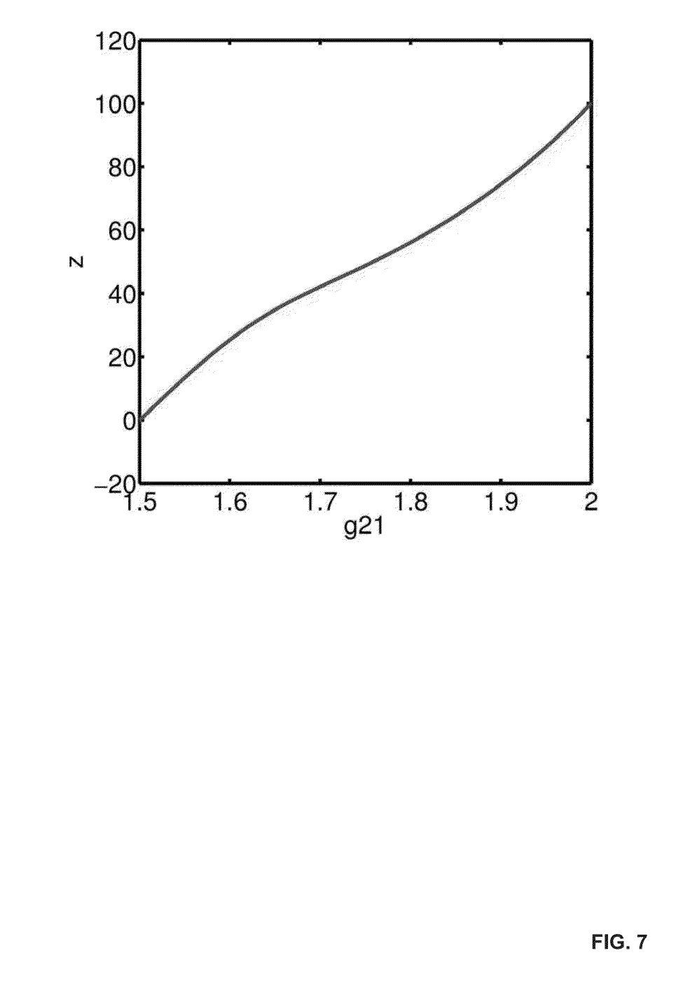

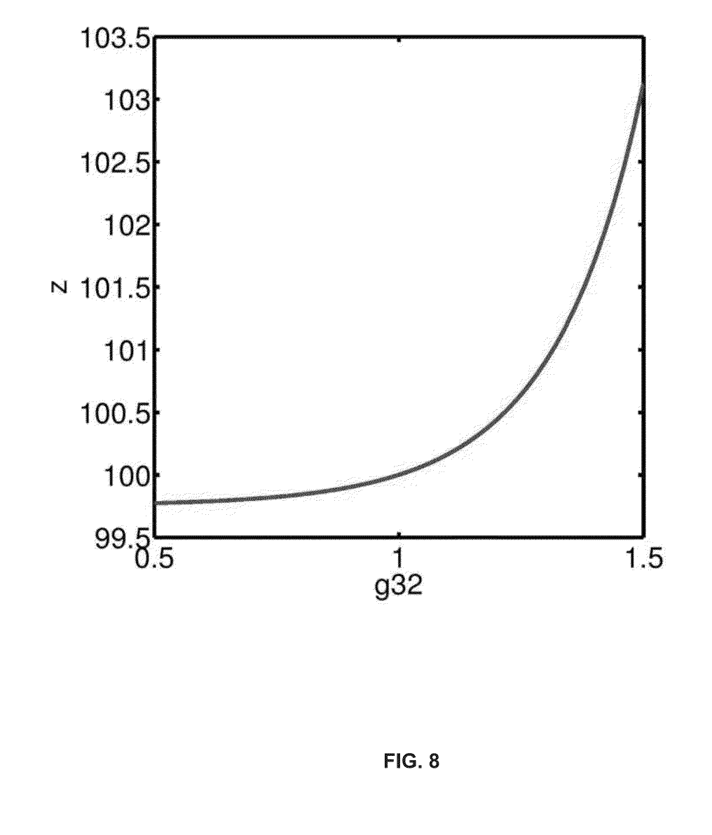

FIG. 6 illustrates the steady state bone volume, Zₛₛ, as a simultaneous function of the effectiveness of osteocyte paracrine signaling on stromal cell differentiation g₂₁, and pre-osteoblast autocrine signaling for pre-osteoblast proliferation g₃₂. FIG. 7 illustrates the steady state bone volume, Zₛₛ, computed as a function of effectiveness of osteocyte paracrine signaling on stromal cell differentiation, g₂₁, with all other parameters held at baseline values with parameters K_{S}=200 cells, α₁=0.5 per day, α₂=0.1 per day, α₃=0.1 per day, β₁=0.1 per day, δ=0.1 per day, β₂=0.1 per day, α₄=0.1 per day, β₃=0.1, g₃₁=1, g₂₂=1 g₃₂=1, f₁₂=1, f₁₄=1, f₂₃=1, g₄₁=1, g₄₂=1, g₄₃=−1, g₄₄=1, f₃₄=1, ε=1 cell, K₁=0.7% volume per day, K₂=0.015545% volume per day. FIG. 8 illustrates that the parameter g₂₁ can be considerably more sensitive to variation than g₃₂, which suggests that the influence of osteocyte signaling, and sclerostin, on stromal cell differentiation into pre-osteoblasts can be key to bone remodeling. In an aspect, the steady state value for bone volume, Zₛₛ can be obtained to correspond to normal bone remodeling for a relatively wide range of g₃₂ values, provided g₂₁ is at or near the baseline value. In an aspect, the significance of the role of osteocyte signaling can be particularly strong with regard to the initiation of a cycle of targeted remodeling, since the steady state values for all cell counts, except osteocytes, is zero, which can mean there is no presence of cells fully committed to either the osteoblast or osteoclast lineage when there is no active bone remodeling.

In an aspect, one or more parameters of the level set function (e.g., level set function (1)) can be associated with an effect of using a drug. As an example, anti-sclerostin drugs can promote bone formation, thereby increase bone mass. Sclerostin can inhibit Wnt/β-catenin, so anti-sclerostin can promote osteoblast differentiation. In addition, Wnt signaling can increase OPG expression, which can inhibit osteoclasts, so anti-sclerostin can be expected to increase OPG release. Sclerostin has been shown to down regulate OPG and increase levels of RANKL. In an aspect, g₂₂ and g₄₄ can represent the effectiveness of sclerostin regulation of osteoblastogenesis and osteoclastogenesis respectively. By modifying g₂₂ and g₄₄, the effects of an anti-sclerostin drug on bone remodeling can be determined in a situation where it is known to be an imbalance in resorption/formation (e.g., abnormal osteoclast activity).

In an aspect, the parameters g₂₂ and g₄₄ can be configured to be time dependent, that is g₂₂(t) and g₄₄(t). The form of the time dependence can be determined by dosing time of the drug, dosing frequency of the drug, half-life of the drug, or combination thereof. Since g₂₂ and g₄₄ are exponents in a power law approximation they can be dimensionless quantities even when they are configured to depend on time. In an aspect, the function g₂₂(t) can be a constant value G₂₂ over a specific time interval t₁ to t₂, and baseline value otherwise. As an example, baseline values can be K_{S}=200 cells, α₁=0.5 per day, α₂=0.1 per day, α₃=0.1 per day, β₁=0.1 per day, δ=0.1 per day, β₂=0.1 per day, α₄=0.1 per day, β₃=0.1, g₃₁=1, g₂₁=2, g₂₂=1, g₃₂=1, f₁₂=1, f₁₄=1, f₂₃=1, g₄₁=1, g₄₂=1, g₄₃=−1, g₄₄=1, f₃₄=1, ε=1 cell, K₁=0.7% volume per day, K₂=0.015545% volume per day. The function g₄₄(t) can be a constant G₄₄ over another time interval t₃ to t₄, and the baseline value otherwise. The constants G₂₂ and G₄₄ can represent perturbation of baseline parameter values for g₂₂ and g₄₄ respectively. In order to get an increase in bone formation, G₂₂ can be set less than the baseline value, and G₄₄ can be set greater than the baseline value. The time intervals t₁ to t₂, and t₃ to t₄ can represent the period of activity of a given dose of a drug (e.g., an anti-sclerostin drug). The time interval t₁ to t₂ can be the time over which the drug (e.g., an anti-sclerostin drug) effects Wnt signaling. The time interval t₃ to t₄ can be the time over which the drug (e.g., an anti-sclerostin drug) effects OPG signaling. In an aspect, time interval t₁ to t₂ and time interval t₃ to t₄ can be the same.

In an aspect, effects of other drugs for treating bone disease such as multiple myeloma can be associated with one or more parameters of the level set function. As an example, the drugs for multiple myeloma treatment can be combination of Velcade® and Dex®; Thalidomide® and Dex®; Lenalidomide® and Dex®; Melphalan® and Prednisone®; Pomalidomide® and Dex®; Carfilzomib® and Dex®; Lenalidomide®, Velcade® and Dex®; Thalidomide®, Velcade® and Dex®; Melphalan®, Velcade® and Prednisone®; Velcade®, Cyclophosphamide® and Dex®; Velcade®, Doxil® and Dex®. In an aspect, parameters in the level set function (1) can be associated with the effects of drug for treating bone disease. As an example, the parameters in the level set function (1) can be determined by bone marrow biopsy data and standard parameter estimation techniques such as Bayesian parameter estimation techniques, Levenberg-Marquardt method or other Quasi-Newton methods for deterministic parameter fitting.

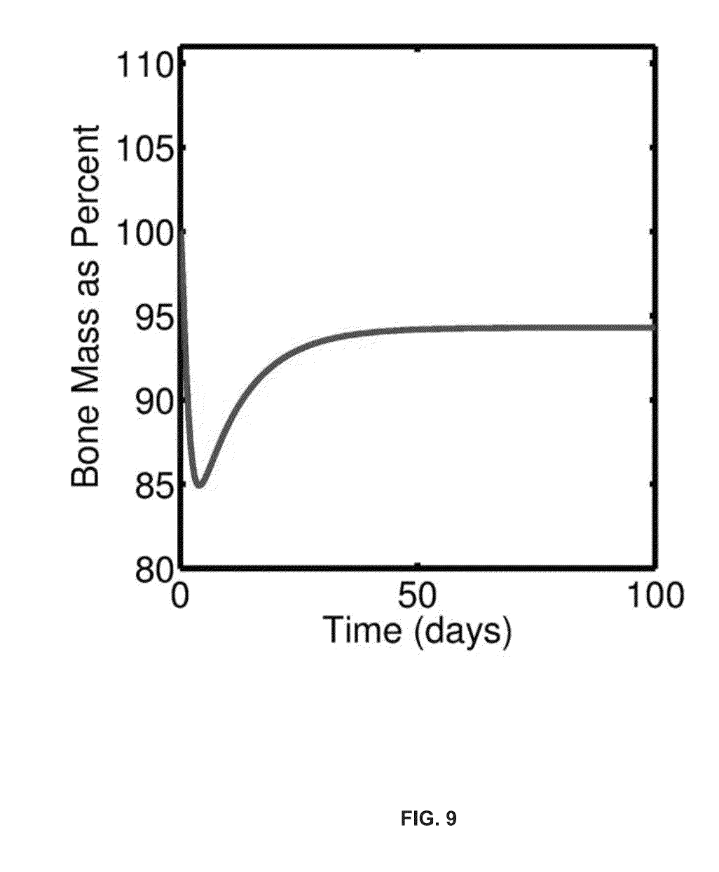

FIG. 9 illustrates change in bone mass as a function of time in a simulated pathological remodeling, wherein there is over resorption and no treatment. This can be modeled by increasing the value of α₄ from its baseline value as α₄=0.1 per day to α₄=0.11 per day. Specifically, K_{S}=200 cells, α₁=0.5 per day, α₂=0.1 per day, α₃=0.1 per day, β₁=0.1 per day, δ=0.1 per day, β₂=0.1 per day, α₄=0.11 per day, β₃=0.1, g₃₁=1, g₂₁=2, g₂₂=1, g₃₂=1, f₁₂=1, f₁₄=1, f₂₃=1, g₄₁=1, g₄₂=1, g₄₃=−1, g₄₄=1, f₃₄=1, ε=1 cell, K₁=0.7% volume per day, K₂=0.015545% volume per day. g₂₂ and g₄₄ is not yet modified in FIG. 9.

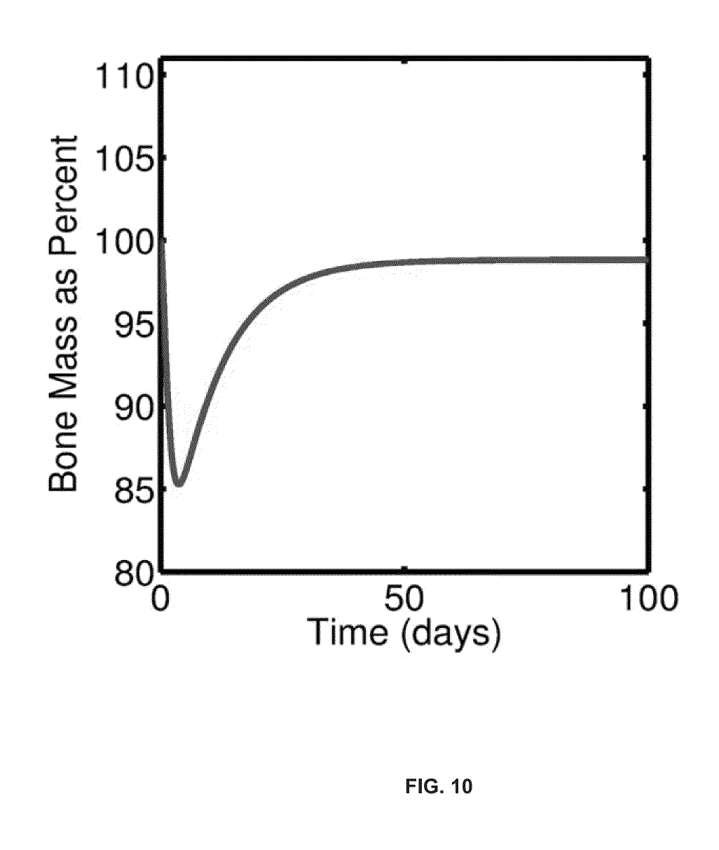

FIG. 10 illustrates results obtained by including treatment with an antisclerostin drug, implemented by modifying the parameters g₂₂ and g₄₄. Treatment with an anti-sclerostin drug can be modeled by modifying the appropriate exponents g₂₂ and g₄₄ in the power law approximation. Specifically, all the parameters in the level set function (1) are the same as those used to obtain the results show in FIG. 9, but g₂₂ and g₄₄ can be modified as functions g₂₂(t) and g₄₄(t) to model the effects of treatment with an anti-sclerostin drug. For example, the time of the anti-sclerostin drug dosing can be associated with g₂₂(t) and g₄₄(t). The closer to initial stages of osteoclastogenesis that a dose is given, the better the response of increased bone formation. This is particularly the case with respect to OPG signaling related parameter g₄₄. As another example, the parameter g₂₂, which can correspond primarily to Wnt signaling, can be sensitive to the perturbations that model dosing with an anti-sclerostin drug. That is, it can take a small decrease in the value over a short period of time for a marked increase in bone formation. In an aspect, the parameters of the level set function (e.g., level set function (1)) can be affected by dosing frequency and half-life of a drug. In this case, all the parameters in the level set function (1) can be time dependent.

In another aspect, the level set function can be set function (2):

$$\begin{matrix}\frac{d C}{d t} = {α}_{1} {C}^{{g}_{11 ( 1 + {r}_{11} \frac{T}{{L}_{T}} )}} {B}^{{g}_{21 ( 1 + {r}_{21} \frac{T}{{L}_{T}} )}} - {β}_{1} C & ( 1 ) \\ \frac{d B}{d t} = {α}_{2} {C}^{\frac{{g}_{12}}{1 + {r}_{12} \frac{T}{{L}_{T}}}} {B}^{( {g}_{22} - \frac{T}{{L}_{T}} {r}_{22} )} - {β}_{2} B & ( 2 ) \\ \frac{d T}{d t} = {γ}_{T} T Log ( \frac{{L}_{T}}{T} ) & ( 3 ) \\ \frac{d Z}{d t} = - {κ}_{1} max \{ 0 , C - \overline{C} \} + {κ}_{2} max \{ 0 , B - \overline{B} \} & ( 4 )\end{matrix}$$

wherein steady-state values of osteoclast C̅ and osteoblast counts B can be used as thresholds for remodeling activity, and wherein

$$\overline{C} = {( \frac{{α}_{1}}{{β}_{1}} )}^{( \frac{1 - {g}_{22} - {r}_{22}}{A} )} {( \frac{{α}_{2}}{{β}_{2}} )}^{( \frac{{g}_{21}}{A} ) ( 1 + {r}_{21} )}$$

$$\overline{C} = {( \frac{{α}_{1}}{{β}_{1}} )}^{( \frac{\frac{{g}_{21}}{1 + {r}_{12}}}{A} )} {( \frac{{α}_{2}}{{β}_{2}} )}^{\frac{1 - {g}_{11} ( 1 + {r}_{11} )}{A}}$$

$$A = ( 1 - {g}_{11} ( 1 + {r}_{11} ) ) ( 1 - {g}_{22} + {r}_{22} ) - ( \frac{{g}_{12}}{1 + {r}_{12}} ) ( {g}_{211} ( 1 + {r}_{21} ) )$$

wherein C is cell counts for osteoclasts, B is cell counts for osteoblasts, T is cell counts for tumor cells, Z is bone volume or bone mass. α₁ is rate of mature osteoblasts embedded into the extra cellular matrix, α₂ is rate of stem cells differentiate into the pre-osteoblast cell in response to signaling molecules produced by osteocytes, β₁ is rate of pre-osteoblast cells differentiate into osteoblast cells, β₂ is apoptosis rate of osteoblast cells, g₁₁ is effectiveness of osteoclast self promotion (e.g., autocrine signaling), g₂₁ is effectiveness of osteoblast promotion of osteoblast cells (e.g., paracrine signaling), g₁₂ is effectiveness of osteoclast promotion of osteoblast (e.g., paracrine signaling), g₂₂ is effectiveness of osteoblast self promotion (e.g., autocrine signaling), r₁₁ is tumor modification of osteoclast self promotion, r₂₁ is tumor modification of osteoblast promotion of osteoclasts, r₁₂ is tumor modification of osteoclast promotion of osteoblasts, r₂₂ is tumor modification of osteoblast self promotion, L_{T} is tumor scaling density, γ_{T} is Gompertz law coefficient, κ₁ is bone loss coefficient, and κ₂ is bone gain coefficient. As an example, α₁=3 per day, β₁=0.2 per day, α₂=4 per day, β₂=0.02 per day, g₁₁=0.5, g₂₁=−0.5, g₁₂=1, g₂₂=0, L_{T}=100, γ_{T}=0.005, κ₁=0.0748, and κ₂=0.0006395. In an aspect, these numbers are chosen to fit a generic remodeling cycle. In the fully functioning prognostic system, the parameters of the level set function (2) can be determined by data from bone marrow biopsy slides of a patient, tumor cytogentic test results of the patient, patient age, patient nutritional state and other clinical information to reflect staging of a disease. The system can be updated continuously for different patients using standard parameter estimation techniques.

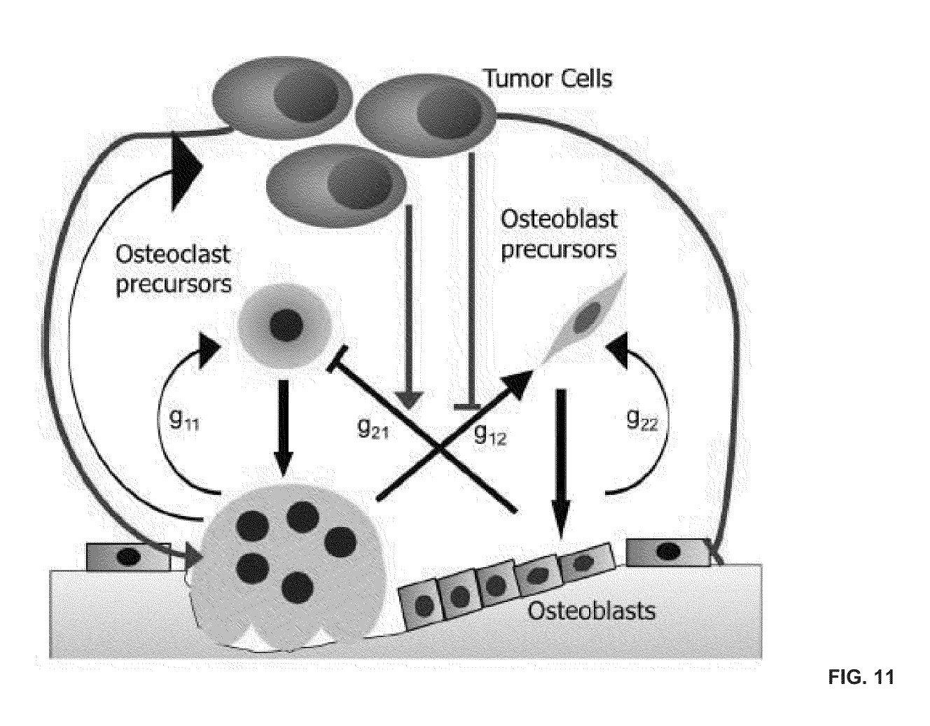

FIG. 11 illustrates an example model of local “microenvironment” interactions. In an aspect, bone remodeling can comprise removal (also called “resorption”) of bone by multinuclear osteoclasts and rebuilding of new bone by osteoblasts. The recruitment of osteoblasts and osteoclasts to a location is interdependent. In multiple myeloma dysregulated bone remodeling, the normal interplay between osteoclasts and osteoblasts can be modified by tumor cells, resulting in net bone loss over time. For example, g₁₁ can represent the effect, in nondimensional terms, of osteoclast autocrine signaling, i.e. the effects of osteoclasts on their own recruitment. g₁₂ can represent the effect, in nondimensional terms, of osteoclast on osteoblast recruitment. g₂₁ can represent the effect, in nondimensional terms, of osteoblast on osteoclast recruitment. g₂₂ can represent the effect, in nondimensional terms, of osteoblast autocrine signaling.

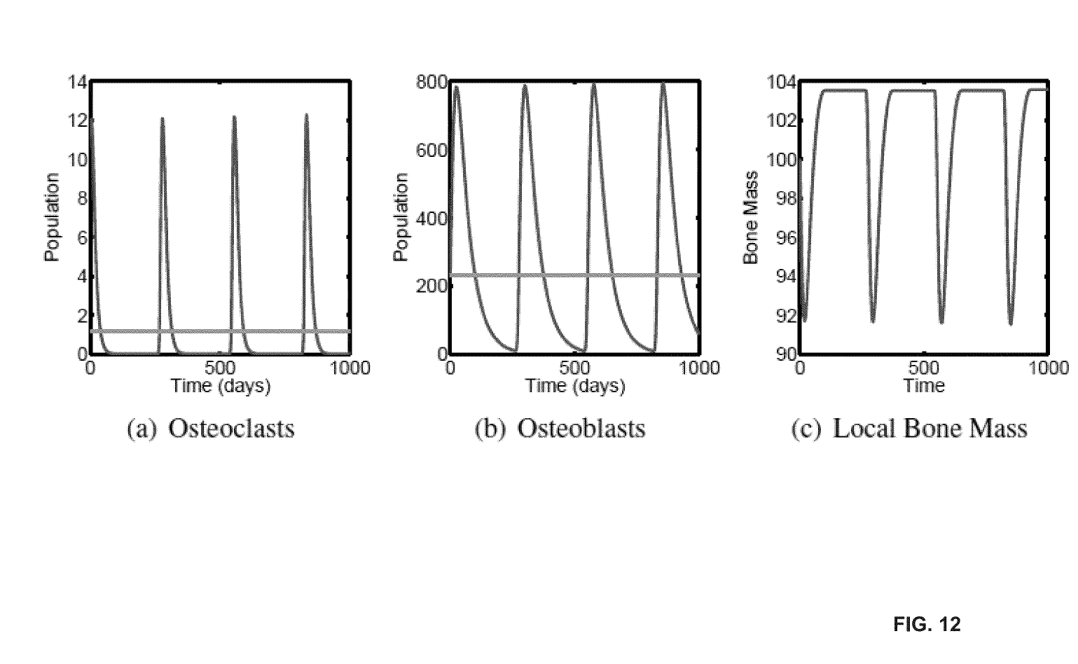

FIG. 12 illustrates results of level set function (2) in the no tumor case, wherein r₁₁=r₁₂=r₂₁=r₂₂=0, α₁=3 per day, β₁=0.2 per day, α₂=4 per day, β₂=0.02 per day, g₁₁=0.5, g₂₁=−0.5, g₁₂=1, g₂₂=0, L_{T}=100, γ_{T}=0.005, κ₁=0.0748, and κ₂=0.0006395. In the case of no tumor, all level set functions components can have regular periodicity. FIG. 12 shows that the level set function (2) can capture normal bone remodeling in a generic sense in the absence of a bone disease.

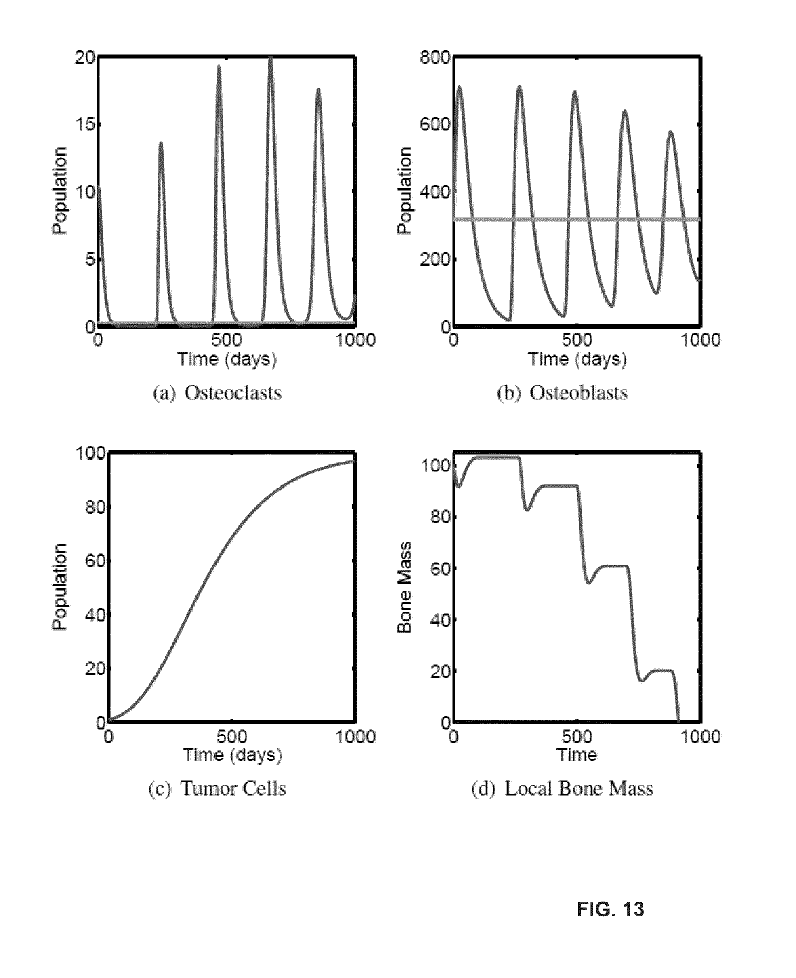

FIG. 13 illustrates results of level set function (2) for multiple myeloma dysregulated autocrine signaling in bone remodeling, wherein r₁₁=0.005, r₁₂=0, r₂₁=0, r₂₂=0.2, α₁=3 per day, β₁=0.2 per day, α₂=4 per day, β₂=0.02 per day, g₁₁=0.5, g₂₁=−0.5, g₁₂=1, g₂₂=0, L_{T}=100, γ_{T}=0.005, κ₁=0.0748, and κ₂=0.0006395. The dynamics show an initial oscillatory increase in osteoclasts and decrease in osteoblasts. As the tumor cell density increases, the bone remodeling system becomes damped and there is an oscillatory decrease in both osteoclast and osteoblast numbers, as well as bone mass. FIG. 13 shows that the level set function (2) can capture bone loss and osteoclast and osteoblast derangement in the presence of multiple myeloma.

In an aspect, the parameters of level set function (2) can be related to effects of a drug treatment. For example, a drug can modify the activity of cell populations such as osteoclast, osteoblast counts, tumor cells. The parameters can be time dependent in the level set function calculations to incorporate time of dosing, dosing frequency and half-life of a drug.

At step 204, bone marrow biopsy data can be received. As an example, the bone marrow biopsy data can be received by computer 101 and stored in imaging data 107 as illustrated in FIG. 1. In an aspect, bone marrow biopsy data can comprise bone and bone marrow histomorphometry data of stained elements in a bone marrow slide. For example, elements associated with parameters of the level set function can be stained and measured, for example, through histological analysis. Specifically, percentages of cells that are expressing RANKL, OPG, and sclerostin can be measured. In another aspect, bone marrow biopsy data can comprise geometry data in a bone marrow slide. For example, geometric data can comprise area of marrow space, area of mineralized bone, area of bone, area of cartilage, area of interstitial fluid, and boundaries of trabeculae. As another example, geometric information can comprise spatial relationships between specific cell types (e.g., osteoclasts, osteoblasts, pre-osteoblasts osteoblasts, plasma cells, myeloma cells, stromal cells, and osteocytes). In another aspect, bone marrow biopsy data can comprise information on cell counts or volume fractions of osteoblasts, osteoclasts, osteocytes, pre-osteoblasts, plasma cells, and stromal cells, stromal cells in a bone marrow biopsy slide. In another aspect, bone marrow biopsy data can reflect progress of a bone disease. For example, balance between osteolysis and osteogenesis can be determined locally by cross-talk between the specific cells types (e.g., osteoclasts, osteoblasts, pre-osteoblasts osteoblasts, plasma cells, myeloma cells, stromal cells, and osteocytes). As another example, an excessive bone resorption can be closely linked to prognosis of a bone disease. As another example, histologic data of osteogenic and osteolytic factors that affect bone density can reflect progression of a bone disease and treatment outcome of a bone disease.

As an example, for histomorphometric data analysis, one or more biopsy cores from a specific patient can be sectioned to more than one obtain 4 adjacent 5 micron-thick sections for quantitative histomorphometry. Specifically, a first section can be stained with standard hematoxylin and cosin (H&E) for the purposes of obtaining morphological data. In an aspect, the H&E section can be analyzed at full resolution to identify histological features of interest. The remaining sections can be stained with immunohistochemical stains specific for receptor activator of nuclear kappa-3 ligand (RANKL), osteoprotegerin (OPG), and sclerostin respectively. For each section, the locations of positively staining cells can be identified, and their locations and absolute counts can be saved. In an aspect, a plurality of bone marrow biopsy slides can be used for collecting bone marrow biopsy data. Each slide can be digitized using a digital histology system.

In an aspect, data from a immunohistochemical section can be spatially registered back to morphological and cell count data from the H&E section by aligning the outer tissue boundary of the biopsy, thereby provide a quantified and spatially mapped data set of relevant cell types (e.g., osteoblasts, osteoclasts, osteocytes, plasma cells, and stromal cells) and their expression of several key signaling proteins (e.g., OPG, RANKL, and Sclerostin). Overall cell density can be computed per total cell, per total tissue area, or per mineralized tissue area, and percentage of cells expressing each signaling protein can be contemplated variables for inclusion in the level set function. Post-hoc calculations of additional variables derived from the quantified histology data can be incorporated into the level set function. For example, proximity of cells expressing a particular signaling protein to a secondary cell type, in particular, proximity of cells between stromal cells and myeloma cells can be calculated through a generalization of the specific models listed to ones using spatially explicit partial differential equations or integro-differential equations. The level set function (2) can represent cell populations that comprising of a field of ordinary differential equations where cells can be recruited globally to each spot inside the marrow.

In an aspect, immunohistochemical sections can be analyzed using a counting routine to analyze histological images. In an aspect, the counting routine can be created to identify particular cell types (e.g. viable vs. necrotic osteocytes in a study of osteonecrosis, number and spatial distribution of chondrocytes in a study of osteoarthritis). In another aspect, the counting routine can be created for identifying and quantifying morphological and cellular features in the H&E stained biopsy cores. By using a combination of edge detection, color-based thresholding and texture analysis, the area of marrow space, area of mineralized bone, area of bone, area of cartilage, area of interstitial fluid, and boundaries of trabeculae can be identified. As an example, within the mineralized bone area, osteocytes and/or empty osteocyte lacunae can be identified using color-based thresholding, ellipse-detecting Hough transform, and cell specific dimension data. Within marrow space, plasma cells, stromal cells, and tumor cells can be identified based on color-based thresholding, ellipse-detecting Hough transform, and cells specific dimension data, with incorporation of absolute color and nuclear aspect ratio. For example, osteoblasts can be identified based on color and aspect ratio along the surfaces of the trabeculae. As another example, osteoclasts can be identified as cells fitting particular morphological criteria, including location in pits (e.g., highly convex regions) along the trabecular boundary. In addition to absolute counts of specific cell (e.g., osteoblasts, osteoclasts, osteocytes), coordinates of the specific cells and trabecular boundaries can be recorded to facilitate calculations of spatial distribution information such as proximity or clustering.

At step 206, a plurality of parameters of the level set function can be determined based on the bone marrow biopsy data. In an aspect, parameters of the level set function can be determined by extracting information from bone marrow biopsy data. In an aspect, parameters can be determined by an automated process. For example, bone marrow biopsy data (e.g., imaging data 107) can be processed in imaging data processing software 106 of the computer 101 to determine the plurality of parameters of a level set function. As an example, histology data such as cell counts, volume fractions of osteoblasts, osteoclasts, osteocytes, pre-osteoblasts, tumor cells, plasma cells, and stromal cells can be processed to determine the plurality of parameters of the level set function. Geometry data such as distribution of osteoblasts, osteoclasts, osteocytes, pre-osteoblasts, tumor cells, plasma cells, and stromal cells, edges of trabeculae, area of mineralized bone, marrow space, and the like can be used to determine parameters of a level set function. As such, the parameters of the level set function (1) and (2) can be exacted from bone marrow biopsy data and determined by standard parameter estimation techniques.

At step 208, bone volume or bone mass Z, cell counts for one or more of, osteoclasts, osteoblasts, pre-osteoblast, osteocytes, and tumor cells can be predicted. For example, by applying the level set function created at step 202 (e.g., level set function (1) or (2)) to the parameters determined at step 206, bone volume or bone mass Z and cell counts for one or more of, osteoclasts C, osteoblasts B, osteocytes S and tumor cells T can be predicted by solving the level set function (e.g., level set function (1) or (2)) using MATLAB or other numerical analysis technics.

At step 210, treatment outcome of a bone disease can be determined based on the bone volume, bone mass, cell counts for osteoclasts, osteoblasts, pre-osteoblasts, osteocytes and tumor cells. In an aspect, the information obtained at step 208 can provide insight into prognosis of a bone disease or outcome of treatment of a bone disease. As an example, bisphosphonate therapy is used to treat multiple myeloma. Bisphosphonate therapy can inhibit osteoclast activity and decrease the incidence of skeletal-related events in multiple myeloma. However, bisphosphonate therapy can have a variety of side effects. In an aspect, information obtained in step 208 can allow physicians to identify patients with varying degrees of derangement in bone remodeling, thereby determined whether an immediate treatment with bisphosphonate therapy is needed for a patient and when to initiate bisphosphonate therapy. As another example, information obtained in step 208 can also be used to determine when to terminate a bisphosphonate therapy based on evidence of increasing derangement in bone homeostasis despite continued bisphosphonate use, thereby decreasing the risk of adverse side effects. Once the model parameters are determined from the patient's bone marrow biopsy data, then a well-defined level set function can be solved. Specifically, the level set functions (e.g., level set function (1), level set function (2)) can be solved to predict of one or more components in the level set functions, particularly the components relating to a disease state.

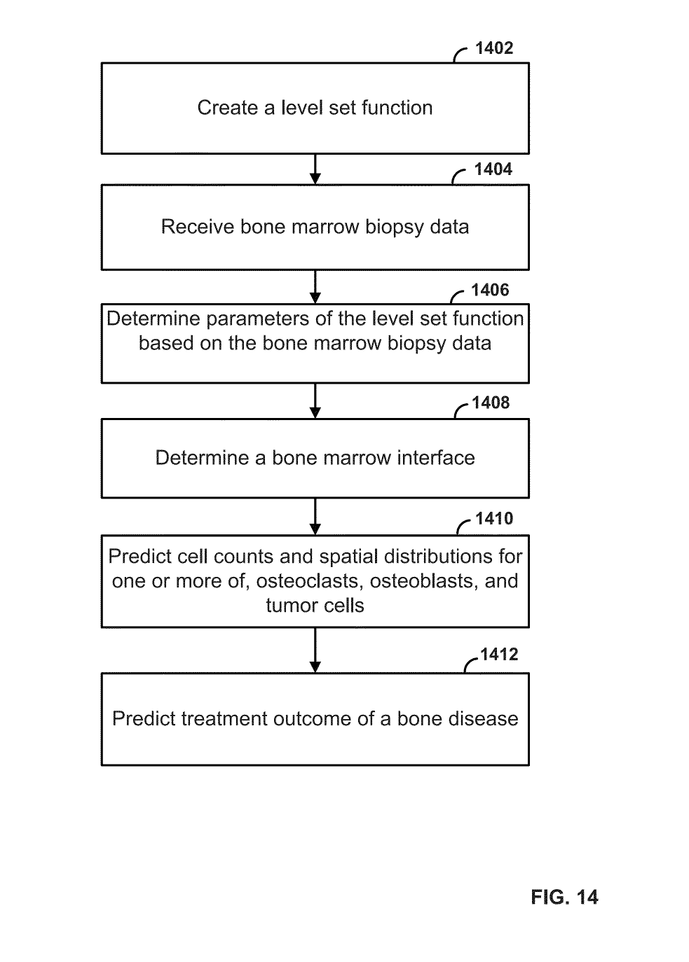

FIG. 14 is a flowchart illustrating another example method for orthopedic treatment and bone disease treatment. At step 1402, a level set function is created. In an aspect, the level set function can be the level set function (3):

$$\begin{matrix}\frac{d C}{d t} = {α}_{1} {C}^{{g}_{11 ( 1 + {r}_{11} \frac{T}{{L}_{T}} )}} {B}^{{g}_{21 ( 1 + {r}_{21} \frac{T}{{L}_{T}} )}} - {β}_{1} C \\ \frac{d B}{d t} = {α}_{2} {C}^{\frac{{g}_{12}}{1 + {r}_{12} \frac{T}{{L}_{T}}}} {B}^{( {g}_{22} - \frac{T}{{L}_{T}} {r}_{22} )} - {β}_{2} B \\ \frac{d T}{d t} = {γ}_{T} T Log ( \frac{{L}_{T}}{T} ) \\ \frac{d φ}{d t} + a ‖ ∇ φ ‖ = 0\end{matrix}$$

wherein,
a(x,t)=−κ₁ max{0,C− C̅}+κ₂ max{0,B− B̅}
C(x,t)=C(x,t),xεΓt
B(x,t)=C(x,t),xεΓt
T(x,t)=C(x,t),xεΓt
φ(x,0)=φ₀(x)
   wherein C is cell counts for osteoclasts, B is cell counts for osteoblasts, T is cell counts for tumor cells, φ is bone marrow interface. α₁ is rate of mature osteoblasts embedded into the extra cellular matrix, α₂ is rate of stem cells differentiate into the pre-osteoblast cell in response to signaling molecules produced by osteocytes, β₁ is rate of pre-osteoblast cells differentiate into osteoblast cells, β₂ is apoptosis rate of osteoblast cells, g₁₁ is effectiveness of osteoclast self promotion (e.g., autocrine signaling), g₂₁ is effectiveness of osteoblast promotion of osteoblast cells (e.g., paracrine signaling), g₁₂ is effectiveness of osteoclast promotion of osteoblast (e.g., paracrine signaling), g₂₂ is effectiveness of osteoblast self promotion (e.g., autocrine signaling). r₁₁ is tumor modification of osteoclast self promotion, r₂₁ is tumor modification of osteoblast promotion of osteoclasts, r₁₂ is tumor modification of osteoclast promotion of osteoblasts, r₂₂ is tumor modification of osteoblast self promotion, L_{T} is tumor scaling density, γ_{T} is Gompertz law coefficient, and α is a velocity term of bone and bone marrow interface. In an aspect, tumor modification can represent how the tumor changes the interaction of the other two types

At step 1404, bone marrow biopsy data can be received. As an example, the bone marrow biopsy data can be received by computer 101 and stored in imaging data 107 as illustrated in FIG. 1. In an aspect, bone marrow biopsy data can come from a patient with a bone disease such as myeloma dysregulated bone disease, an orthopedics condition such as articular cartilage damage, or a combination thereof. In an aspect, bone marrow biopsy data can comprise histomorphometry data of stained elements in a bone marrow slide. For example, elements associated with parameters of the level set function can be stained and measured, for example, through histological analysis. Specifically, percentages of cells that are expressing RANKL, OPG, and sclerostin can be measured. In another aspect, bone marrow biopsy data can comprise geometry data in a bone marrow slide. For example, geometric data can comprise area of marrow space, area of mineralized bone, area of bone, area of cartilage, area of interstitial fluid, and boundaries of trabeculae. As another example, geometric information can comprise spatial relationships between specific cell types (e.g., osteoclasts, osteoblasts, pre-osteoblasts osteoblasts, plasma cells, myeloma cells, stromal cells, and osteocytes). In another aspect, bone marrow biopsy data can comprise information on cell counts or volume fractions of osteoblasts, osteoclasts, osteocytes, pre-osteoblasts, plasma cells, and stromal cells, stromal cells in a bone marrow biopsy slide. In another aspect, bone marrow biopsy data can reflect progress of a bone disease. For example, balance between osteolysis and osteogenesis can be determined locally by cross-talk between the specific cells types (e.g., osteoclasts, osteoblasts, pre-osteoblasts osteoblasts, plasma cells, myeloma cells, stromal cells, and osteocytes). As another example, an excessive bone resorption can be closely linked to prognosis of a bone disease. As another example, histologic data of osteogenic and osteolytic factors that affect bone density can reflect progression of a bone disease and treatment outcome of a bone disease.

As an example, for histomorphometric data analysis, one or more biopsy cores from a specific patient can be sectioned to obtain 4 adjacent 5 micron-thick sections for quantitative histomorphometry. Specifically, a first section can be stained with standard hematoxylin and eosin (H&E) for the purposes of obtaining morphological data. In an aspect, the H&E section can be analyzed at full resolution to identify histological features of interest. The remaining sections can be stained with immunohistochemical stains specific for receptor activator of nuclear kappa-β ligand (RANKL), osteoprotegerin (OPG), and sclerostin respectively. For each section (RANKL, OPG, sclerostin), the locations of positively staining cells can be automatically identified, and their locations and absolute counts can be saved. In an aspect, a plurality of bone marrow biopsy slides can be used for bone marrow biopsy data. Each slide can be digitized using a digital histology system.

In an aspect, data from a immunohistochemical section can be spatially registered back to morphological and cell count data from the H&E section by aligning the outer tissue boundary of the biopsy, thereby provide a quantified and spatially mapped data set of relevant cell types (e.g., osteoblasts, osteoclasts, osteocytes, plasma cells, and stromal cells) and their expression of several key signaling proteins (e.g., OPG, RANKL and Sclerostin). Overall cell density can be computed per total cell, per total tissue area, or per mineralized tissue area, and percentage of cells expressing each signaling protein can be contemplated variables for inclusion in the level set function. Post-hoc calculations of additional variables derived from the quantified histology data can be incorporated into the level set function. In an aspect, proximity of cells expressing a particular signaling protein to a secondary cell type, for example, proximity of cells, particularly between stromal cells and myeloma cells can be calculated through a generalization of the level set functions (e.g., level set function (3)) using spatially explicit partial differential equations or integro-differential equations. The level set function (e.g., level set function (3)) can calculate cell populations that comprises of a field of ordinary differential equations where cells can be recruited globally to each spot inside the marrow.

In an aspect, immunohistochemical sections can be analyzed using counting routines to analyze histological images. In an aspect, the counting routine can be created to identify particular cell types (e.g. viable vs. necrotic osteocytes in a study of osteonecrosis, number and spatial distribution of chondrocytes in a study of osteoarthritis). In another aspect, the counting routine can be created for identifying and quantifying morphological and cellular features in the H&E stained biopsy cores. By using a combination of edge detection, color-based thresholding and texture analysis, the area of marrow space, area of mineralized bone, area of bone, area of cartilage, area of interstitial fluid, and boundaries of trabeculae can be identified. As an example, within the mineralized bone area, osteocytes and/or empty osteocyte lacunae can be identified using color-based thresholding, an ellipse-detecting Hough transform, and cell specific dimension data. Within marrow space, plasma cells, stromal cells, and tumor cells can also be identified based on color-based thresholding, ellipse-detecting Hough transform, and cells specific dimension data, with incorporation of absolute color and nuclear aspect ratio. For example, osteoblasts can be identified based on color and nuclear aspect ratio along the surfaces of the trabeculae. As another example, osteoclasts can be identified as cells fitting particular morphological criteria, including location in pits (e.g., highly convex regions) along the trabecular boundary. In addition to absolute counts of specific cells (e.g., osteoblasts, osteoclasts, osteocytes), coordinates of the specific cells and trabecular boundaries can be recorded to facilitate calculations of spatial distribution information such as proximity or clustering.

At step 1406, a plurality of parameters of the level set function based on the bone marrow biopsy data can be determined. In an aspect, the plurality of parameters of the level set function can be determined by extracting information from bone marrow biopsy data. For example, tumor scaling density L_{T} can be determined based on the bone marrow biopsy data comprises determining. As another example, tumor modification of bone cells (e.g., tumor modification of osteoclast self promotion r₁₁, tumor modification of osteoblast promotion of osteoclasts r₂₁, tumor modification of osteoclast promotion of osteoblasts r₁₂, tumor modification of osteoblast self promotion r₂₂) can be determined based on the bone marrow biopsy data. In an aspect, the plurality of parameters such as r₁₁, r₁₂, r₂₁ r₂₂ L_{T} can be determined based on bone marrow biopsy data using standard parameter estimation techniques.

In an aspect, one or more parameters can be determined by an automated process. For example, bone marrow biopsy data can be processed in imaging data processing software 106 of the computer 101 to determine parameters of the level set function. Specifically, histology data such as cell counts, volume fractions of osteoblasts, osteoclasts, osteocytes, pre-osteoblasts, plasma cells, and stromal cells, can be processed to determine parameters in the level set function (3). Geometry data such as distribution of osteoblasts, osteoclasts, osteocytes, pre-osteoblasts, plasma cells, and stromal cells, tumor cells, edges of trabeculae, area of marrow space, area of mineralized bone, area of bone, area of cartilage, area of interstitial fluid, and the like can be used to determine parameters in the level set function (3).

At step 1408, a bone marrow interface can be determined. In an aspect, determining the bone marrow interface can comprise determining one or more of, area of marrow space, area of mineralized bone, area of bone, area of cartilage, area of interstitial fluid, and boundaries of trabeculae. For example, morphological and cellular features associated with marrow space, area of mineralized bone, area of bone, area of cartilage, area of interstitial fluid, and boundaries of trabeculae can be identified and quantified in one or more H&E stained biopsy cores by using a combination of edge detection, color-based thresholding and texture analysis. As an example, within the mineralized bone regions, osteocytes and/or empty osteocyte lacunae can be identified using color-based thresholding, ellipse-detecting Hough transform, and cells specific dimension data. Within marrow space, plasma cells, stromal cells, and tumor cells can also be identified based on color-based thresholding, ellipse-detecting Hough transform, and cells specific dimension data, with incorporation of absolute color and nuclear aspect ratio.

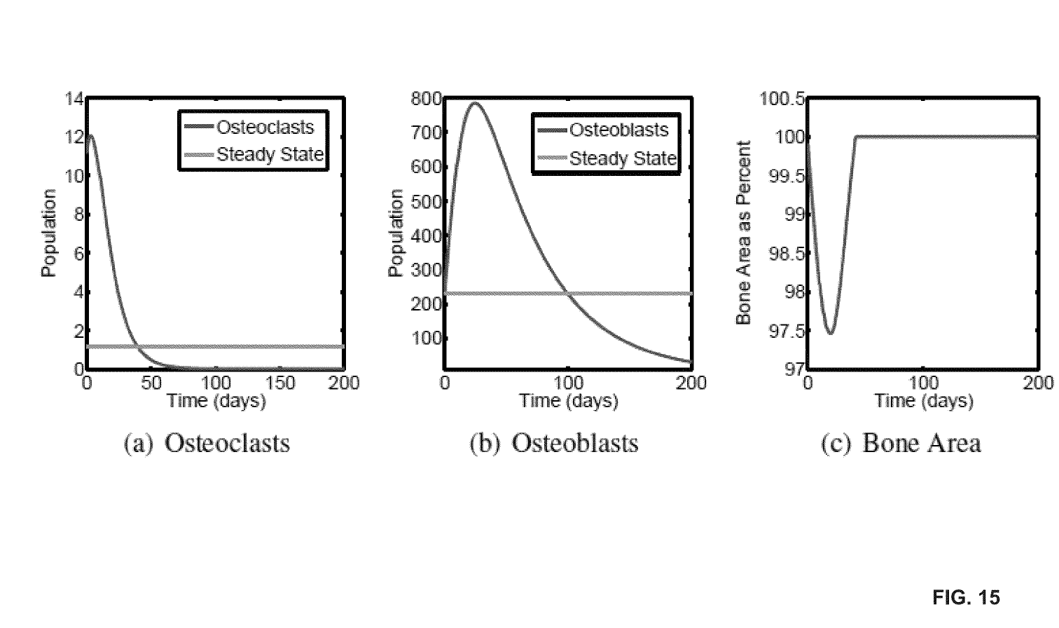

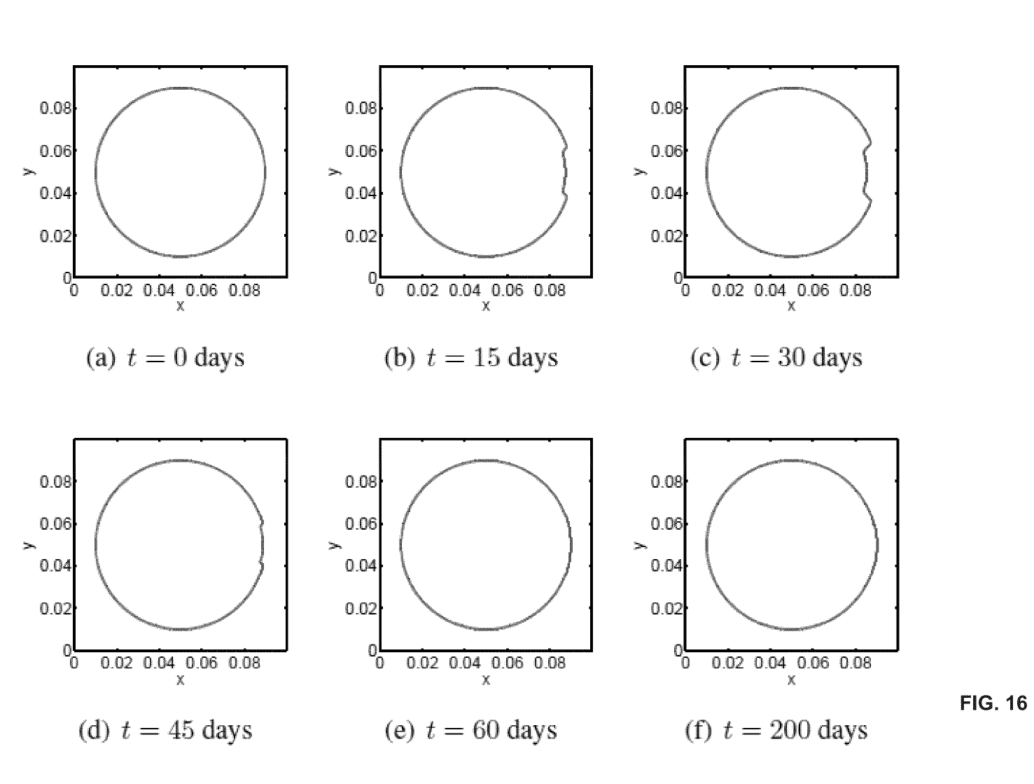

At step 1410, cell counts and spatial distributions for one or more of, osteoclasts, osteoblasts, and tumor cells can be determined. FIG. 15 and FIG. 16 illustrate computational results for the level set function (3) for normal bone remodeling in one remodeling cycle. Parameters are the same as the level set function (2), with the replacement of scaling constants with κ₁=0.0748, and κ₂=0.0006395. Specifically, FIG. 15 illustrates aggregation across space of the dynamics under normal bone remodeling (r₁₁=r₁₂=r₂₁=r₂₂=0) of the spatially explicit model. FIG. 16 illustrates bone and bone marrow interface snapshots in time in the case of normal bone remodeling. FIG. 16 begins with an idealized circular geometry for the bone and bone marrow interface (the interior of the circle is trabecular bone). After an area of bone is resorbed and reformed, the bone and bone marrow interface returns to its original shape, absent of anything that would indicate an osteolytic lesion.

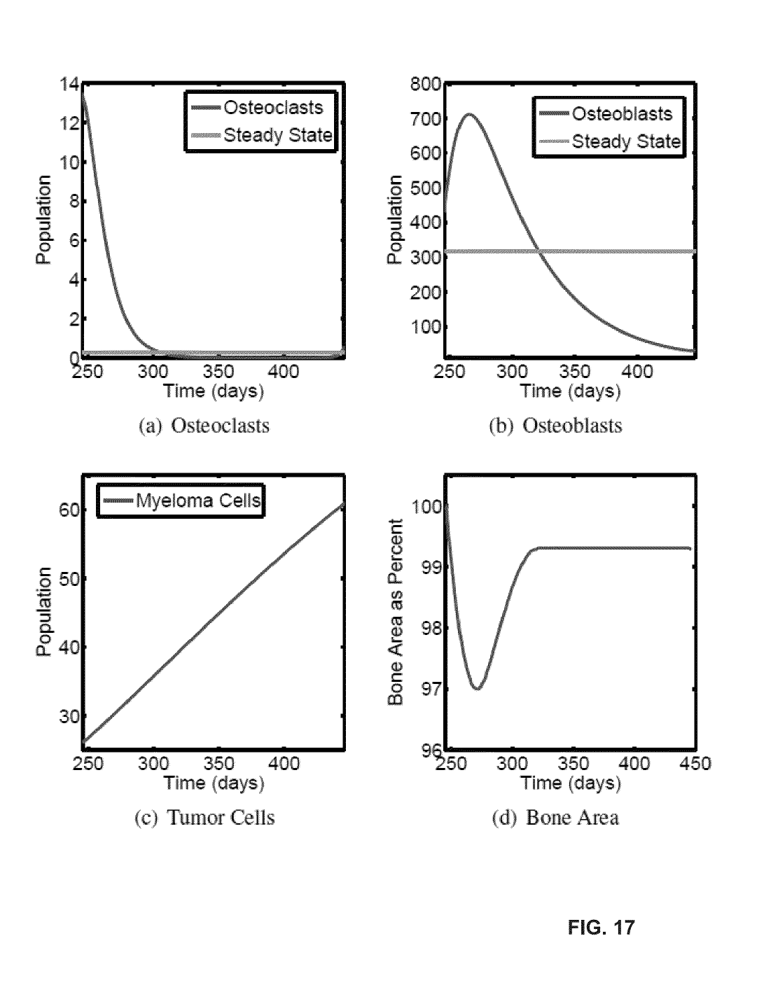

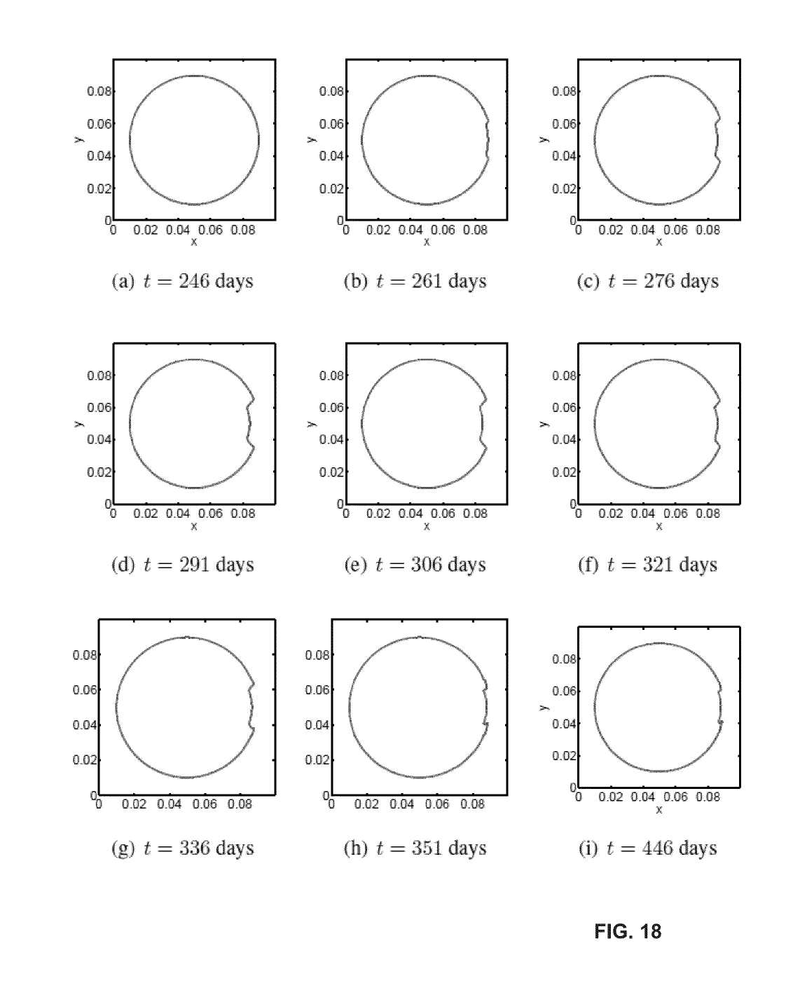

FIG. 17 and FIG. 18 illustrate computational results for the level set function (3) for myeloma dysregulated bone remodeling in one remodeling cycle. Specifically, FIG. 17 illustrates aggregation across space of the dynamics under multiple myeloma dysregulated bone remodeling (r₁₁=0.005, r₁₂=0, r₂₁=0, r₂₂=0.2) of the spatially explicit model. FIG. 18 illustrates bone and bone marrow interface snapshots in time in the case of multiple myeloma dysregulated bone remodeling. FIG. 18 begins with an idealized circular geometry for the bone and bone marrow interface (the interior of the circle is trabecular bone). The effects of multiple myeloma can manifest themselves as a net loss of bone mass at the remodeling site over a remodeling cycle. The section of remodeled bone is not fully restored and development of an osteolytic lesion can be seen.

In an aspect, using an idealized geometry for the set level set function (3) can help visualize the formation of an osteolytic lesion more clearly than using a more complicated geometry. The use of an idealized circular geometry can show clearly the formation of an osteolytic lesion in computations with tumor load (FIG. 18), and the absence of such lesions in the case of normal bone remodeling (FIG. 17). In an aspect, actual geometries can be used to replace an idealized circular geometry.

At step 1412, treatment outcome of a bone disease can be predicted based on the cell counts and spatial distributions for one or more of, osteoclasts, osteoblasts, and tumor cells. In an aspect, information obtained in step 1410 can provide insight into the pathological relationship between tumor cells and the sounding bone marrow environment, and improved understanding of morbidity of a bone disease. As an example, bisphosphonate therapy is used to treat multiple myeloma. In an aspect, bisphosphonate therapy can inhibit osteoclast activity and decrease the incidence of skeletal-related events in multiple myeloma. However, bisphosphonate therapy can have a variety of side effects. In an aspect, information obtained in step 1410 can allow physicians to identify patients with varying degrees of derangement in bone remodeling, thereby determined whether an immediate treatment with bisphosphonate therapy is needed for a patient and when to initiate bisphosphonate therapy. As another example, information obtained in step 1410 can also be used to determine when to terminate a bisphosphonate therapy based on evidence of increasing derangement in bone homeostasis despite continued bisphosphonate use, thereby decreasing the risk of adverse side effects.

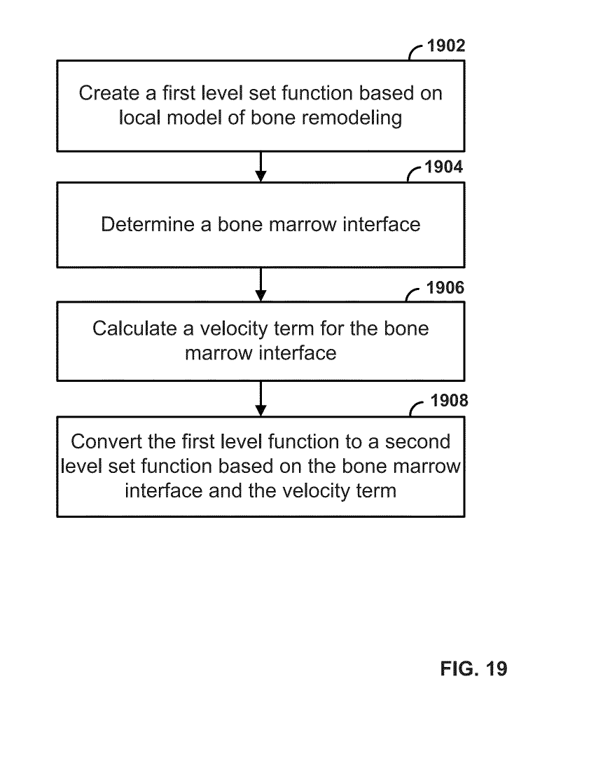

FIG. 19 is a flowchart illustrating another example method for orthopedic treatment and bone disease treatment. At step 1902, a level set function can be created based on a local model of bone remodeling. In an aspect, the level set function can be created by mathematical power-law formalism of biochemical systems theory (BST) for biochemical interactions in bone remodeling described in FIG. 3 and FIG. 11. In an aspect, the level set function can be the level set function (2):

$$\begin{matrix}\frac{d C}{d t} = {α}_{1} {C}^{{g}_{11 ( 1 + {r}_{11} \frac{T}{{L}_{T}} )}} {B}^{{g}_{21 ( 1 + {r}_{21} \frac{T}{{L}_{T}} )}} - {β}_{1} C \\ \frac{d B}{d t} = {α}_{2} {C}^{\frac{{g}_{12}}{1 + {r}_{12} \frac{T}{{L}_{T}}}} {B}^{( {g}_{22} - \frac{T}{{L}_{T}} {r}_{22} )} - {β}_{2} B \\ \frac{d T}{d t} = {γ}_{T} T Log ( \frac{{L}_{T}}{T} ) \\ \frac{d Z}{d t} = - {κ}_{1} max \{ 0 , C - \overline{C} \} + {κ}_{2} max \{ 0 , B - \overline{B} \} \\ wherein \\ \overline{C} = {( \frac{{α}_{1}}{{β}_{1}} )}^{( \frac{1 - {g}_{22} - {r}_{22}}{A} )} {( \frac{{α}_{2}}{{β}_{2}} )}^{( \frac{{g}_{21}}{A} ) ( 1 + {r}_{21} )} \\ \overline{C} = {( \frac{{α}_{1}}{{β}_{1}} )}^{( \frac{\frac{{g}_{21}}{1 + {r}_{12}}}{A} )} {( \frac{{α}_{2}}{{β}_{2}} )}^{\frac{1 - {g}_{11} ( 1 + {r}_{11} )}{A}} \\ A = ( 1 - {g}_{11} ( 1 + {r}_{11} ) ) ( 1 - {g}_{22} + {r}_{22} ) - ( \frac{{g}_{12}}{1 + {r}_{12}} ) ( {g}_{211} ( 1 + {r}_{21} ) )\end{matrix}$$

wherein C is cell counts for osteoclasts, B is cell counts for osteoblasts, T is cell counts for tumor cells, φ is bone marrow interface, α₁ is rate of mature osteoblasts embedded into the extra cellular matrix, α₂ is rate of stem cells differentiate into the pre-osteoblast cell in response to signaling molecules produced by osteocytes, β₁ is rate of pre-osteoblast cells differentiate into osteoblast cells, β₂ is apoptosis rate of osteoblast cells, g₁₁ is effectiveness of osteoclast self promotion (e.g., autocrine signaling), g₂₁ is effectiveness of osteoblast promotion of osteoblast cells (e.g., paracrine signaling), g₁₂ is effectiveness of osteoclast promotion of osteoblast (e.g., paracrine signaling), g₂₂ is effectiveness of osteoblast self promotion (e.g., autocrine signaling), r₁₁ is tumor modification of osteoclast self-promotion, r₂₁ is tumor modification of osteoblast promotion of osteoclasts, r₁₂ is tumor modification of osteoclast promotion of osteoblasts, r₂₂ is tumor modification of osteoblast self-promotion, L_{T} is tumor scaling density, γ_{T} is Gompertz law coefficient, and α is a velocity term of bone marrow interface φ.

At step 1904, a bone marrow interface can be determined. In an aspect, determining the bone marrow interface can comprise determining one or more of area of marrow space, area of mineralized bone, area of bone, area of cartilage, area of interstitial fluid, and boundaries of trabeculae. For example, morphological and cellular features associated with marrow space, area of mineralized bone, area of bone, area of cartilage, area of interstitial fluid, and boundaries of trabeculae can be identified and quantified in one or more H&E stained biopsy cores by using a combination of edge detection, color-based thresholding and texture analysis. As an example, within the mineralized bone regions, osteocytes and/or empty osteocyte lacunae can be identified using color-based thresholding, ellipse-detecting Hough transform, and cells specific dimension data. Within marrow space, plasma cells, stromal cells, and tumor cells can also be identified based on color-based thresholding, ellipse-detecting Hough transform, and cells specific dimension data, with incorporation of absolute color and nuclear aspect ratio.

At step 1906, a velocity term for the bone marrow interface can be calculated. As an example, a sharp interface between bone and bone marrow can be denoted by Γt, wherein Γt⊂R² and wherein R² can represent a standard x,y coordinate system real plane Γt can be associated with a level set function φ(x,t) by the relationship Γt={x=(x1,x2)εR²:φ(x,t)=0}. In an aspect, a level set can represent an interface implicitly as the zero level set of a higher dimensional function. For example, a circle of radius 1 centered at (0; 0) can be described explicitly using one independent variable as (cos t, sin t) for 0≦t<2π or implicitly using two independent variables by setting φ(x1, x2)=1−x₁²−x₂². In an aspect, there can be advantages in moving an interface defined implicitly using a level set function over moving an interface defined by an explicit representation. Moreover, a level set function can provide a natural extension of the local model by replacing the term for change of bone mass in the level set function (2) with the velocity term of the interface by respective scaling constants. For example, the scaling constants can be used to convert the concept of local bone mass to change in the position of the level set function through fits to the bone marrow biopsy data. The introducing of the velocity term can enable bone remodeling to depend on time and space with dependent variables, specifically,

C(x,t)=osteoclast counts density at position xεR² and time t,
B(x,t)=osteoblast counts density at position xεR² and time t,
T(x,t)=tumor cell counts density at position xεR² and time t,
a(x,t)=velocity of the bone marrow interface at position xεΓt⊂R² and time t.

In an aspect, the velocity term for the bone marrow interface can be calculated through calculating one or more of deterministic integro-differential equations, stochastic integro-differential equations or combination thereof.

In an aspect, the rate of change of bone mass

$$\frac{d Z ( t )}{d t}$$

in the level set function (2) can be replaced by a velocity term a(x,t) for the bone marrow interface φ. In an aspect, the equation for bone loss in level set function (2) can be equivalent to a normal velocity a of the bone marrow interface φ.

At step 1908, the first level set function can be converted to a second level set function based on the bone marrow interface φ and the velocity term a. In an aspect, the second level set function can be associated with a spatial model of the bone remodeling. When a local model for bone remodeling represented by the first level set function (e.g., level set function (2)) is converted to a spatial scale bone remodeling represented by the second level set function (e.g., level set function (3)), the rate of change of bone mass

$$\frac{d Z ( t )}{d t}$$

in the first level set function can be replaced by a velocity term a(x, t) for the bone marrow interface φ. In the second level set function, the osteoclast counts C(x, y, t) and osteoblast counts B(x, y, t) at time t and at a specific point in space (x, y) can include precursors, and their equations can be coupled to a Gompertz law for tumor load (T).

In an aspect, the second level set function can be:

$$\begin{matrix}\frac{d C}{d t} = {α}_{1} {C}^{{g}_{11 ( 1 + {r}_{11} \frac{T}{{L}_{T}} )}} {B}^{{g}_{21 ( 1 + {r}_{21} \frac{T}{{L}_{T}} )}} - {β}_{1} C \\ \frac{d B}{d t} = {α}_{2} {C}^{\frac{{g}_{12}}{1 + {r}_{12} \frac{T}{{L}_{T}}}} {B}^{( {g}_{22} - \frac{T}{{L}_{T}} {r}_{22} )} - {β}_{2} B \\ \frac{d T}{d t} = {γ}_{T} T Log ( \frac{{L}_{T}}{T} ) \\ \frac{d φ}{d t} + a ‖ ∇ φ ‖ = 0\end{matrix}$$

wherein,
a(x,t)=−κ₁ max{0,C− C̅}+κ₂ max{0,B− B̅}
C(x,t)=C(x,t),xεΓt
B(x,t)=C(x,t),xεΓt
T(x,t)=C(x,t),xΓΓt
φ(x,0)=φ₀(X)
   wherein C is cell counts for osteoclasts, B is cell counts for osteoblasts, T is cell counts for tumor cells, φ is bone marrow interface. α₁ is rate of mature osteoblasts embedded into the extra cellular matrix, α₂ is rate of stem cells differentiate into the pre-osteoblast cell in response to signaling molecules produced by osteocytes, β₁ is rate of pre-osteoblast cells differentiate into osteoblast cells, β₂ is apoptosis rate of osteoblast cells, g₁₁ is effectiveness of osteoclast self promotion (e.g., autocrine signaling), g₂₁ is effectiveness of osteoblast promotion of osteoblast cells (e.g., paracrine signaling), g₁₂ is effectiveness of osteoclast promotion of osteoblast (e.g., paracrine signaling), g₂₂ is effectiveness of osteoblast self promotion (e.g., autocrine signaling), r₁₁ is tumor modification of osteoclast self promotion, r₂₁ is tumor modification of osteoblast promotion of osteoclasts, r₁₂ is tumor modification of osteoclast promotion of osteoblasts, r₂₂ is tumor modification of osteoblast self promotion, L_{T} is tumor scaling density, γ_{T} is Gompertz law coefficient, and a is a velocity term of bone and bone marrow interface. In an aspect, the parameters in the second level set function (e.g., level set function (3)) can be determined based on bone marrow biopsy data. In another aspect, the second level set function can be used to predict an orthopedic treatment and/or a bone disease treatment, as described in FIG. 14.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method comprising:
creating a level set function;
receiving bone marrow biopsy data;
determining a plurality of parameters of the level set function based on the bone marrow biopsy data; and
predicting bone volume, bone mass, and cell counts for one or more of, osteoclasts, osteoblasts, pre-osteoblasts, osteocytes, plasma cells, stromal cells, and tumor cells by the calculating the level set function according to the plurality of parameters based on the bone marrow biopsy data.

2. The method of claim 1, wherein the level set function is based on mathematical power law formalism of biochemical systems theory (BST).

3. The method of claim 1, wherein the bone marrow biopsy data comprises bone and bone marrow histomorphometry data.

4. The method of claim 1, further comprising predicting treatment outcome of a bone disease, based on one or more of bone volume, bone mass, cell counts for osteoclasts, osteoblasts, pre-osteoblasts, osteocytes, plasma cells, stromal cells, and tumor cells.

5. The method of claim 1, wherein the pluralities of parameters are associated with bone marrow biopsy data.

6. The method of claim 1, wherein the plurality of parameters are associated with interactions between osteoclasts, osteoblasts, pre-osteoblasts, and osteocytes osteoclasts, osteoblasts, pre-osteoblasts, osteocytes, tumor cells, plasma cells, and stromal cells.

7. The method of claim 1, wherein one or more of the plurality of parameters of the level set function are associated with an effect of a drug on bone remodeling.

8. The method of claim 7, wherein one or more of the plurality of parameters associated with the effect of the drug is a function of dosing time of the drug, dosing frequency of the drug, half-life of the drug, or combination thereof.

9. A method comprising:
creating a level set function;
receiving bone marrow biopsy data;
determining a plurality of parameters of the level set function based on the bone marrow biopsy data;
determining a bone marrow interface; and
predicting cell counts and spatial distributions for one or more of, osteoclasts, osteoblasts, and tumor cells.

10. The method of claim 9, wherein the level set function is based on mathematical power law formalism of biochemical systems theory (BST).

11. The method of claim 9, wherein the bone marrow biopsy data comprises bone and bone marrow histomorphometry data.

12. The method of claim 9, wherein determining a bone marrow interface comprises determining one or more of area of marrow space, area of mineralized bone, area of bone, area of cartilage, area of interstitial fluid, and boundaries of trabeculae.

13. The method of claim 9, further comprising predicting treatment outcome of a bone disease based on the cell counts and spatial distribution for one or more of, osteoclasts, osteoblasts, and tumor cells.

14. The method of claim 9, wherein the plurality of parameters are associated with bone marrow biopsy data.

15. The method of claim 9, wherein the plurality of parameters are associated with interactions between osteoclasts, osteoblasts, pre-osteoblasts, and osteocytes, tumor cells, plasma cells, and stromal cells.

16. The method of claim 9, wherein one or more parameters of the level set function is associated with an effect of a drug on bone remodeling.

17. The method of claim 16, wherein one or more parameters associated with the effect of the drug is a function of one or more of, dosing time of the drug, dosing frequency of the drug, half-life of the drug, or combination thereof.

18. The method of claim 9, wherein determining parameters of the level set function based on the bone marrow biopsy data comprises determining tumor scaling density.

19. The method of claim 9, wherein determining parameters of the level set function based on the bone marrow biopsy data comprises determining tumor modification of bone cells.

20. The method of claim 19, wherein the bone cells comprises osteoblasts and osteoclasts.

21. The method of claim 9, wherein bone marrow biopsy data are data from a patient with a bone disease, an orthopedics condition, or a combination thereof.

22. A method comprising:
creating a first level set function based on a local model of bone remodeling;
determining a bone marrow interface;
calculating a velocity term for the bone marrow interface; and
converting the first level set function to a second level set function based on the bone marrow interface and the velocity term.

23. The method of claim 22, wherein the second level set function is associated with a spatial model of bone remodeling.

24. The method of claim 22, wherein the bone marrow interface is determined by bone marrow biopsy data.

25. The method of claim 22, wherein the bone marrow biopsy data is morphology data.

26. The method of claim 22, wherein determining a bone marrow interface comprises determining one or more of area of marrow space, area of mineralized bone, area of bone, area of cartilage, area of interstitial fluid, and boundaries of trabeculae.
